(19) United States
(12) Patent Application Publication
(10) Pub. No.: US 2010/0016275 A1
(43) Pub. Date: Jan. 21, 2010

(54) NOVEL COMPOUND 395

(76) Inventors: PREMJI MEGHANI, Loughborough (GB); ANDREW JAMES ROBBINS, Loughborough (GB); JEFFREY PAUL STONEHOUSE, Loughborough (GB)

Correspondence Address: FISH & RICHARDSON P.C., P.O BOX 1022, MINNEAPOLIS, MN, 55440-1022, US

(21) Appl. No.: 12/503,433

(22) Filed: Jul. 15, 2009

Related U.S. Application Data
(60) Provisional application No. 61/081,213, filed on Jul. 16, 2008.

(51) Int. Cl. A61K 31/506; C07D 403/12; C07D 239/24; A61K 31/505

(52) U.S. Cl. 514/210.2; 544/317; 514/274

(57) ABSTRACT

A compound of formula (1)

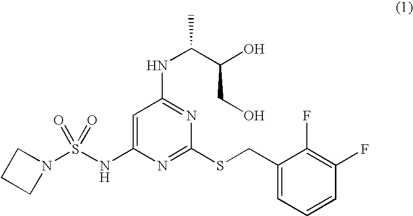

and pharmaceutically acceptable salts thereof for use in the treatment of chemokine mediated diseases and conditions.

This application claims the benefit under 35 U.S.C. §119(e) of U.S. Provisional Application No. 61/081213 filed on 16 Jul. 2008, which is incorporated herein by reference in its entirety.

The present invention relates to certain heterocyclic compounds, processes and intermediates used in their preparation, pharmaceutical compositions containing them and their use in therapy.

Chemokines play an important role in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved cysteine motif. At the present time, the chemokine superfamily comprises three groups exhibiting characteristic structural motifs, the C—X—C, C—C and C—X₃—C families. The C—X—C and C—C families have sequence similarity and are distinguished from one another on the basis of a single amino acid insertion between the NH-proximal pair of cysteine residues. The C—X₃—C family is distinguished from the other two families on the basis of having a triple amino acid insertion between the NH-proximal pair of cysteine residues.

The C—X—C chemokines include several potent chemoattractants and activators of neutrophils such as interleukin-8 (IL-8) and neutrophil-activating peptide 2 (NAP-2).

The C—C chemokines include potent chemoattractants of monocytes and lymphocytes but not neutrophils. Examples include human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

The C—X₃—C chemokine (also known as fractalkine) is a potent chemoattractant and activator of microglia in the central nervous system (CNS) as well as of monocytes, T cells, NK cells and mast cells.

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family. These receptors represent good targets for drug development since agents which modulate these receptors would be useful in the treatment of disorders and diseases such as those mentioned above.

In our PCT patent application WO 2004/011443 we disclose pyrimidinyl sulfonamide derivatives for use as modulators of chemokine receptors.

The present invention now provides the compound of formula (1) and

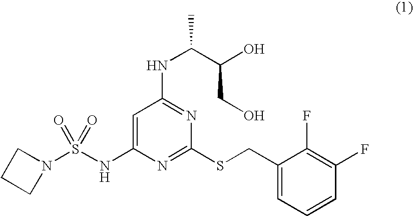

pharmaceutically acceptable salts thereof Such compound is not anticipated by reference to the compounds disclosed in WO-2004/011443, there being always at least two structural differences. In addition we have found that the compound of formula (1) shows an improved pharmacological profile when compared with such compounds. Specifically is the compound of formula (1) has at least one improved pharmacological property as set out hereinafter. Whilst we do not wish to be limited by theoretical considerations the improved pharmacological profile of the compound of formula 1 is anticipated to produce a longer duration of action in man. In one aspect of the invention it may allow for once or twice daily dosing of the compound of formula 1.

The synthesis of optically active forms may be carried out by standard techniques of organic chemistry well known in the art, for example by synthesis from optically active starting materials or by resolution of a racemic form (e.g. See Enantioselective Synthesis of fully protected anti 3-amino-2-hydroxy butyrates; Tetrahedron Asymmetry; 1995, vol 6, no 9 pp 2329-2342). Similarly, the above-mentioned activity may be evaluated using the standard laboratory techniques referred to hereinafter.

Within the present invention it is to be understood that the compound of formula (1) or a salt or solvate thereof may exhibit the phenomenon of tautomerism and that the formulae drawings within this specification can represent only one of the possible tautomeric forms. It is to be understood that the invention encompasses any tautomeric form and mixtures thereof and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein.

It is also to be understood that the compound of formula (1) and salts thereof can exist in solvated as well as unsolvated forms such as, for example, hydrated forms. It is to be understood that the invention encompasses all such solvated or hydrated forms.

The present invention relates the compound of formula (1) as hereinbefore defined as well as to the salts thereof. Salts for use in pharmaceutical compositions will be pharmaceutically acceptable salts, but other salts may be useful in the production of the compound of formula (1) and their pharmaceutically acceptable salts. Pharmaceutically acceptable salts of the invention may include basic addition salts of the compound of formula (1) as hereinbefore defined which are sufficiently basic to form such salts. Such salts may be formed with an inorganic or organic base which affords a pharmaceutically acceptable cation. Such salts with inorganic or organic bases include for example an alkali metal salt, such as a sodium or potassium salt, an alkaline earth metal salt such as a calcium or magnesium salt, or an organic amine salt, for example a salt with tris-(2-hydroxyethyl)amine, diethanolamine, or ethanolamine.

The present invention further provides a process for the preparation of the compound of formula (1) as defined above which comprises: (a) treating a compound of formula (2a)

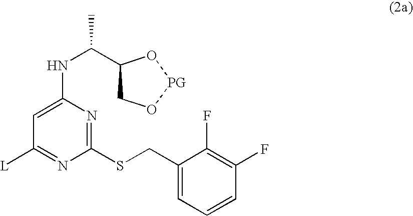

wherein PG is a protecting group or two separate hydrogen atoms and L is a leaving group such as halogen with the sulfonamide (2c):

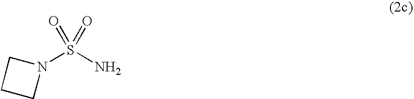

in the presence of a suitable base, catalyst and solvent, and optionally thereafter (i) or (ii) in any order:
i) removing any protecting groups;
ii) forming a salt

Reaction of compounds of formula (2a) with the sulfonamide (2c) can be carried out in the presence of a suitable catalyst and heated thermally or by microwaves.

Examples of suitable bases include metal (bi)carbonates such as those from cesium, potassium, lithium or sodium or metal phosphates such as those from lithium, sodium or potassium (for example potassium phosphate (K₃PO₄)) or trialkylamines such as triethylamine or N,N-di-isopropylethylamine. Most conveniently cesium carbonate is used. Suitable solvents include toluene and ethers such as anisole, tetrahydrofuran, 2-methyltetrahydrofuran, 1,4-dioxane, glyme and diglyme or esters such as n-butylacetate or isopropylacetate. Conveniently 1,4-dioxane is used. The reaction can be performed at temperatures between 10° C. and 120° C., Conveniently at 105° C. Examples of suitable catalysts include a suitable palladium(0) source such as palladium tris(dibenzylideneacetone)dipalladium(0) (Pd₂(dba)₃), or tetrakistriphenylphosphinepalladium (Pd(Ph₃)₄) (either in 0.01-0.5 mol equivalents) in the presence of a suitable ligand such as (9,9-dimethyl-9H-xanthene-4,5-diyl)bis[diphenyl-phosphine (Xantphos), or 2-dicyclohexyl-phosphino-2′-(N,N-dimethylamino)biphenyl or 2-dicyclohexyl-phosphino-2′,4′,6′-tri-isopropyl, 1,1′-biphenyl (XPHOS) (either in 0.01-0.5 mol equivalents). Conveniently the catalyst combination is tris(dibenzylideneacetone)dipalladium(0) (Pd₂(dba)₃) with 2-dicyclohexyl-phosphino-2′,4′,6′-tri-isopropyl,1,1′-biphenyl (Xphos) in 0.01-0.5 mol equivalents in 1,4-dioxane at 105° C. with cesium carbonate as the base.

Suitable protecting groups (PG) include both acyclic and cyclic compounds. Examples of acyclic protecting groups include benzyl, para-nitrobenzyl or para-methoxylbenzyl. Conveniently PG is cyclic. Examples of suitable cyclic protecting groups include cyclohexylidenes, cyclopentylidenes and acetonides. Conveniently the acetonide protecting group is used.

or alternatively;

(b) treating a compound of formula (2b)

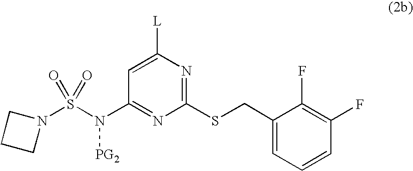

wherein PG₂ is a protecting group and L is a leaving group such as halogen with an amine of the formula (2d)

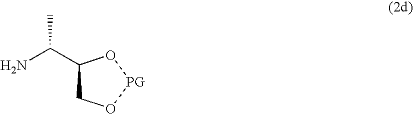

wherein PG is a suitable protecting group or two separate hydrogen atoms, in the presence of a suitable base and solvent, and optionally thereafter (i) and/or (ii) in any order:
i) removing any protecting groups;
ii) forming a salt

Reaction of compounds of formula (2b) with the amine (2d) can be carried out in the presence of a suitable base, solvent and heated thermally or by microwaves

Examples of suitable bases include metal (bi)carbonates such as sodium, potassium cesium or trialkylamines such as triethylamine or N,N-di-isopropylethylamine. Conveniently sodium bicarbonate is used.

Suitable solvents include N,N-dimethylamides, 1-methyl-2-pyrolidinone, toluene and ethers such as anisole, tetrahydrofuran, 2-methyltetrahydrofuran 1,4-dioxane, glyme, diglyme and esters such as n-butylacetate or isopropylacetate and alkylnitriles such acetonitrile or butyronitrile. Conveniently acetonitrile is used.

The reaction can be performed at temperatures between 10° C. and 120° C.

Compounds of formula (2a) can be prepared from compounds of formula (3)

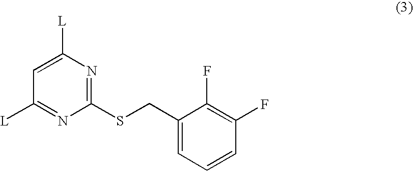

wherein L is a leaving group such as halogen, by treatment with the amine (2d) wherein PG is a protecting group or two separate hydrogen atoms, in the presence of a suitable base and solvent.

Examples of suitable bases include metal (bi)carbonates such as sodium, potassium cesium or trialkylamines such as triethylamine or N,N-di-isopropylethylamine. Conveniently sodium bicarbonate is used.

Suitable solvents include N,N-dimethylamides, 1-methyl-2-pyrolidinone, ethers such as tetrahydrofuran, 2-methyltetrahydrofuran 1,4-dioxane, glyme and diglyme and esters such as butylacetate or isopropylacetate and alkylnitriles such acetonitrile or butyronitrile. Conveniently acetonitrile is used.

The reaction can be performed at temperatures between 10° C. and 120° C., conveniently at 100° C.

Compounds of formula (2b) wherein L is a leaving group such as halogen and PG₂ is either a suitable protecting group or hydrogen, may be prepared by reaction of compounds of formula (3), wherein L is a leaving group such as halogen with the sulfonamide (2c) in the presence of a suitable base, solvent with or without a suitable catalyst heated thermally or by microwaves,

and optionally thereafter (i) or (ii) in any order:
i) adding any protecting groups;
ii) converting the compound of formula (2b) into a further compound of formula (2b).

Examples of suitable bases include the alkali metal hydrides such as sodium or potassium, or metal alkoxides such as lithium, sodium or potassium-tert-butoxide, alkali metal hexamethyldisilazides such as lithium, sodium or potassium-hexamethyldisilazide, or metal carbonates such as sodium, potassium ceasium. Suitable solvents include acetonitrile, tetrahydrofuran, 2-methyltetrahydrofuran 1,4-dioxane, glyme and diglyme. The temperature of the reaction can be performed between 0° C. and 120° C. Examples of suitable catalysts include a suitable palladium(0) source such as tetrakistriphenylphosphinepalladium (Pd(Ph₃)₄) or tris(dibenzylideneacetone)dipalladium(0) (Pd₂(dba)₃) in the presence of a suitable ligand such as (9,9-dimethyl-9H-xanthene-4,5-diyl)bis[diphenyl-phosphine (Xantphos), or 2-dicyclohexyl-phosphino-2′-(N,N-dimethylamino)biphenyl or 2-dicyclohexyl-phosphino-2′,4′,6′-tri-isopropyl,1,1′-biphenyl (XPHOS).

Examples of convenient protecting groups (PG₂) include ethers such as trimethylsilylmethyl ethers (SEM) by alkylation using [2-(chloromethoxy)ethyl](trimethyl)silane or para-methoxybenzyl (PMB) group by alkylation using para-methoxybenzylchloride.

Compounds of formula (3) wherein L is halogen may be prepared from compounds of formula (3) wherein L is a hydroxy group by reaction with a halogenating agent such as phosphorous oxychloride with or without a suitable solvent. The reaction may be carried out in the presence or absence of N,N-dimethylaniline. Suitable solvents include toluene, xylenes, acetonitrile, tetrahydrofuran, 2-methyltetrahydrofuran 1,4-dioxane, glyme and diglyme.

The reaction can be performed at temperatures between 90° C.-150° C.

Compounds of formula (3) wherein L is a hydroxy group may be prepared from compounds of formula (4);

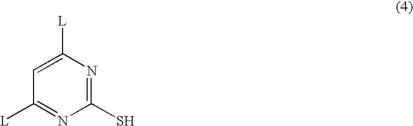

wherein L is a hydroxy group by reaction with 1-(bromomethyl)-2,3-difluorobenzene, in the presence of a suitable base and solvent.

Examples of suitable bases include the alkali metal hydroxides such as lithium, sodium, potassium or metal (bi)carbonates such as lithium, sodium, potassium, cesium or metal acetates such as lithium, sodium, potassium or cesium or metal alkoxides such as lithium, sodium potassium tert-butoxide. Suitable solvents include water, N,N-dimethylamides, 1-methyl-2-pyrolidinone, ethers such as tetrahydrofuran, 2-methyltetrahydrofuran, 1,4-dioxane, glyme and diglyme and alcohols such as methanol, ethanol and tert-butanol or acetonitrile. Conveniently sodium acetate in methanol and water mixtures thereof at 30-60° C. is used. More conveniently sodium acetate in acetonitrile and water mixtures thereof at 40° C. is used.

Compounds of formulae (4), wherein L is a hydroxy group, (2c) and (2d), wherein PG is either a protecting group such as an acetonide or cyclohexylidene or two separate hydrogen atoms are either prepared using procedures described herein, are commercially available, are well known in the literature or may be easily prepared using known techniques.

In each of the process variants outlined above for preparation of compounds of the formula (1) or a pharmaceutically acceptable salt, solvate, or in vivo hydrolysable ester thereof, each of the stated convenient or suitable materials or reaction conditions represents an individual and distinct aspect of the present invention.

It will be appreciated by those skilled in the art that in the processes of the present invention certain functional groups such as hydroxyl or amino groups in the starting reagents or intermediate compounds may need to be protected by protecting groups. Thus, the preparation of the compounds of formula (1) may involve, at an appropriate stage, the removal of one or more protecting groups. The protection and deprotection of functional groups is fully described in ‘Protective Groups in Organic Chemistry’, edited by J. W. F. McOmie, Plenum Press (1973), and ‘Protective Groups in Organic Synthesis’, 2nd edition, T. W. Greene & P. G. M. Wuts, Wiley-Interscience (1991).

Examples of convenient leaving groups are provided in standard chemistry textbooks such as “Organic Chemistry” by Jonathan Clayden et al, published by Oxford University Press (3^{rd} Edn 2005) They include halogen, mesylate and tosylate groups. Halogen, such as chlorine or bromine, conveniently chlorine is a convenient leaving group.

The compound of formula (1) above may be converted to a pharmaceutically acceptable salt or solvate thereof, as discussed above. The salt is conveniently a basic addition salt. The compound of formula (1) has activity as a pharmaceutical, in particular as a modulator of chemokine receptor (especially CXCR2) activity, and may be used in the treatment (therapeutic or prophylactic) of conditions/diseases in human and non-human animals which are exacerbated or caused by excessive or unregulated production of chemokines. Examples of such conditions/diseases include, wherein each condition/disease is taken independently or in any combination thereof:

(1) the respiratory tract—obstructive airways diseases including chronic obstructive pulmonary disease (COPD); asthma, such as bronchial, allergic, intrinsic, extrinsic and dust asthma, particularly chronic or inveterate asthma (e.g. late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia;

(2) bone and joints—rheumatoid arthritis, osteoarthritis seronegative spondyloarthropathies (including ankylosing spondylitis, psoriatic arthritis and Reiter's disease), Behchet's disease, Sjogren's syndrome and systemic sclerosis;

(3) skin—psoriasis, atopical dermatitis, contact dermatitis and other eczmatous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia areata and vernal conjunctivitis;

(4) gastrointestinal tract—Coeliac disease, proctitis, eosinopilic gastro-enteritis, mastocytosis, Crohn's disease, ulcerative colitis, indeterminate colitis, microscopic colitis, inflammatory bowel disease, irritable bowel syndrome, non-inflammatory diarrhea, food-related allergies which have effects remote from the gut, e.g., migraine, rhinitis and eczema;

(5) central and peripheral nervous system—Neurodegenerative diseases and dementia disorders, e.g. Alzheimer's disease, amyotrophic lateral sclerosis and other motor neuron diseases, Creutzfeldt-Jacob's disease and other prion diseases, HIV encephalopathy (AIDS dementia complex), Huntington's disease, frontotemporal dementia, Lewy body dementia and vascular dementia; polyneuropathies, e.g. Guillain-Barre syndrome, chronic inflammatory demyelinating polyradiculoneuropathy, multifocal motor neuropathy, plexopathies; CNS demyelination, e.g. multiple sclerosis, acute disseminated/haemorrhagic encephalomyelitis, and subacute sclerosing panencephalitis; neuromuscular disorders, e.g. myasthenia gravis and Lambert-Eaton syndrome; spinal disorders, e.g. tropical spastic paraparesis, and stiff-man syndrome: paraneoplastic syndromes, e.g. cerebellar degeneration and encephalomyelitis; CNS trauma; migraine; and stroke.

(6) other tissues and systemic disease—atherosclerosis, Acquired Immunodeficiency Syndrome (AIDS), lupus erythematosus, systemic lupus, erythematosus, Hashimoto's thyroiditis, type I diabetes, nephrotic syndrome, eosinophilia fascitis, hyper IgE syndrome, lepromatous leprosy, and idiopathic thrombocytopenia pupura; post-operative adhesions, and sepsis.

(7) allograft rejection—acute and chronic following, for example, transplantation of kidney, heart, liver, lung, bone marrow, skin and cornea; and chronic graft versus host disease;

(8) cancers—especially non-small cell lung cancer (NSCLC), malignant melanoma, prostate cancer and squamous sarcoma, and tumour metastasis, non melanoma skin cancer and chemoprevention metastases;

(9) diseases—in which angiogenesis is associated with raised CXCR2 chemokine levels (e.g. NSCLC, diabetic retinopathy);

(10) cystic fibrosis;

(11) burn wounds & chronic skin ulcers;

(12) reproductive diseases—for example disorders of ovulation, menstruation and implantation, pre-term labour, endometriosis;

(13) re-perfusion injury—in the heart, brain, peripheral limbs and other organs, inhibition of atherosclerosis.

Thus, the present invention provides the compound of formula (1), or a pharmaceutically-acceptable salt, solvate or an in vivo hydrolysable ester thereof, as hereinbefore defined for use in therapy.

Conveniently the compound of the invention is used to treat diseases in which the chemokine receptor belongs to the CXC chemokine receptor subfamily, more conveniently the target chemokine receptor is the CXCR2 receptor.

Particular conditions which can be treated with the compound of the invention are cancer, diseases in which angiogenesis is associated with raised CXCR2 chemokine levels, and inflammatory diseases such as asthma, allergic rhinitis, COPD, rheumatoid arthritis, psoriasis, inflammatory bowel diseases, osteoarthritis or osteoporosis. Each condition/disease listed above when taken independently or in any combination represents an independent embodiment of the invention.

The compound of the invention may also be used to treat diseases in which the chemokine receptor belongs to the CCR chemokine receptor subfamily, more conveniently the target chemokine receptor is the CCR2b receptor.

In a further aspect, the present invention provides a compound of formula (1), or a pharmaceutically acceptable salt, solvate or in vivo hydrolysable ester thereof, as hereinbefore defined for use as a medicament.

In a still further aspect, the present invention provides the use of the compound of formula (1), or a pharmaceutically acceptable salt, solvate or in vivo hydrolysable ester thereof, as hereinbefore defined for use as a medicament for the treatment of human diseases or conditions in which modulation of chemokine receptor activity is beneficial.

In a still further aspect, the present invention provides the use of the compound of formula (1), or a pharmaceutically acceptable salt, solvate or in vivo hydrolysable ester thereof, as hereinbefore defined for use as a medicament for the treatment of asthma, allergic rhinitis, cancer, COPD, rheumatoid arthritis, psoriasis, inflammatory bowel diseases, osteoarthritis or osteoporosis.

In a further aspect, the present invention provides the use of the compound of formula (1), or a pharmaceutically acceptable salt or solvate thereof, as hereinbefore defined in the manufacture of a medicament for use in therapy.

In a still further aspect, the present invention provides the use of the compound of formula (1), or a pharmaceutically acceptable salt or solvate thereof, as hereinbefore defined in the manufacture of a medicament for the treatment of human diseases or conditions in which modulation of chemokine receptor activity is beneficial.

In a still further aspect, the present invention provides the use of the compound of formula (1), or a pharmaceutically acceptable salt or solvate thereof, as hereinbefore defined in the manufacture of a medicament for the treatment of asthma, allergic rhinitis, cancer, COPD, rheumatoid arthritis, psoriasis, inflammatory bowel diseases, osteoarthritis or osteoporosis.

In the context of the present specification, the term “therapy” also includes “prophylaxis” unless there are specific indications to the contrary. The terms “therapeutic” and “therapeutically” should be construed accordingly.

The invention still further provides a method of treating a chemokine mediated disease wherein the chemokine binds to a chemokine (especially CXCR2) receptor, which comprises administering to a patient a therapeutically effective amount of the compound of formula, or a pharmaceutically acceptable salt or solvate as hereinbefore defined.

The invention also provides a method of treating an inflammatory disease, especially asthma, allergic rhinitis, COPD, rheumatoid arthritis, psoriasis, inflammatory bowel diseases, osteoarthritis or osteoporosis, in a patient suffering from, or at risk of, said disease, which comprises administering to the patient a therapeutically effective amount of a compound of formula (1), or a pharmaceutically acceptable salt or solvate thereof, as hereinbefore defined.

For the above-mentioned therapeutic uses the dosage administered will, of course, vary with the compound employed, the mode of administration, the treatment desired and the disorder indicated.

The compound of formula (1) and pharmaceutically acceptable salts or solvates thereof may be used on its own but will generally be administered in the form of a pharmaceutical composition in which formula (1) compound/salt/solvate (active ingredient) is in association with a pharmaceutically acceptable adjuvant, diluent or carrier. Depending on the mode of administration, the pharmaceutical composition will conveniently comprise from 0.05 to 99% w (per cent by weight), more Conveniently from 0.05 to 80% w, still more Conveniently from 0.10 to 70% w, and even more conveniently from 0.10 to 50% w, of active ingredient, all percentages by weight being based on total composition.

The present invention also provides a pharmaceutical composition comprising the compound of formula (1), or a pharmaceutically acceptable salt or solvate thereof, as hereinbefore defined, in association with a pharmaceutically acceptable adjuvant, diluent or carrier.

The invention further provides a process for the preparation of a pharmaceutical composition of the invention which comprises mixing the compound of formula (1), or a pharmaceutically acceptable salt or solvate thereof, as hereinbefore defined, with a pharmaceutically acceptable adjuvant, diluent or carrier. The pharmaceutical compositions may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally. Conveniently the compounds of the invention are administered orally.

In addition to their use as therapeutic medicines, the compounds of formula (1) and their pharmaceutically acceptable salts or solvate are also useful as pharmacological tools in the development and standardisation of in vitro and in vivo test systems for the evaluation of the effect of chemokine modulation activity in labatory animals such as cats, dogs, rabbits, monkeys, rats and mice, as part of the search for new therapeutic agents.

The invention further relates to combination therapies wherein a compound of formula (I) or a pharmaceutically acceptable salts or solvate thereof, or a pharmaceutical composition or formulation comprising a compound of formula (I) is administered concurrently or sequentially with therapy and/or an agent for the treatment of any one of asthma, allergic rhinitis, cancer, COPD, rheumatoid arthritis, psoriasis, inflammatory is bowel disease, irritable bowel syndrome, osteoarthritis or osteoporosis.

In particular, for the treatment of the inflammatory diseases rheumatoid arthritis, psoriasis, inflammatory bowel disease, irritable bowel syndrome, COPD, asthma and allergic rhinitis the compounds of the invention may be combined with agents such as TNF-α inhibitors such as anti-TNF monoclonal antibodies (such as Remicade, CDP-870 and D₂.E₇.) and TNF receptor immunoglobulin molecules such as Etanercept (Enbrel), non-selective COX-1/COX-2 inhibitors (such as piroxicam, diclofenac), propionic acids such as naproxen, flubiprofen, fenoprofen, ketoprofen and ibuprofen), fenamates (such as mefenamic acid, indomethacin, sulindac, apazone), pyrazolones (such as phenylbutazone), salicylates (such as aspirin), COX-2 inhibitors (such as meloxicam, celecoxib, rofecoxib, valdecoxib and etoricoxib) low dose methotrexate, lefunomide; ciclesonide; hydroxychloroquine, d-penicillamine, auranofin or parenteral or oral gold. For inflammatory bowel disease and irritable bowel disorder further convenient agents include sulphasalazine and 5-ASAs, topical and systemic steroids, immunomodulators and immunosuppressants, antibiotics, probiotics and anti-integrins.

The present invention still further relates to the combination of the compound of the invention together with a leukotriene biosynthesis inhibitor, 5-lipoxygenase (5-LO) inhibitor or 5-lipoxygenase activating protein (FLAP) antagonist such as zileuton; ABT-761; fenleuton; tepoxalin; Abbott-79175; Abbott-85761; N-(5-substituted)-thiophene-2-alkylsulfonamides; 2,6-di-tert-butylphenol hydrazones; methoxytetrahydropyrans such as Zeneca ZD-2138; the compound SB-210661; pyridinyl-substituted 2-cyanonaphthalene compounds such as L-739,010; 2-cyanoquinoline compounds such as L-746,530; indole and quinoline compounds such as MK-591, MK-886, and BAY x 1005.

The present invention still further relates to the combination of the compound of the invention together with a receptor antagonist for leukotrienes LTB.sub4., LTC.sub4., LTD.sub4., and LTE.sub4. selected from the group consisting of the phenothiazin-3-ones such as L-651,392; amidino compounds such as CGS-25019c; benzoxalamines such as lo ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of the compound of the invention together with a PDE4 inhibitor including inhibitors of the isoform PDE4D.

The present invention still further relates to the combination of the compound of the invention together with a antihistaminic H.sub1. receptor antagonists such as cetirizine, loratadine, desloratadine, fexofenadine, astemizole, azelastine, and chlorpheniramine.

The present invention still further relates to the combination of the compound of the invention together with a gastroprotective H₂ receptor antagonist.

The present invention still further relates to the combination of the compound of the invention together with an α₁- and α₂-adrenoceptor agonist vasoconstrictor sympathomimetic agent, such as propylhexedrine, phenylephrine, phenylpropanolamine, pseudoephedrine, naphazoline hydrochloride, oxymetazoline hydrochloride, tetrahydrozoline hydrochloride, xylometazoline hydrochloride, and ethylnorepinephrine hydrochloride.

The present invention still further relates to the combination of the compound of the invention together with anticholinergic agents such as ipratropium bromide; tiotropium bromide; oxitropium bromide; pirenzepine; and telenzepine.

The present invention still further relates to the combination of the compound of the invention together with a β₁- to β₄-adrenoceptor agonists such as metaproterenol, isoproterenol, isoprenaline, albuterol, salbutamol, formoterol, salmeterol, terbutaline, orciprenaline, bitolterol mesylate, and pirbuterol; or methylxanthanines including theophylline and aminophylline; sodium cromoglycate; or muscarinic receptor (M1, M2, and M3) antagonist.

The present invention still further relates to the combination of the compound of s the invention together with an insulin-like growth factor type I (IGF-1) mimetic.

The present invention still further relates to the combination of the compound of the invention together with an inhaled glucocorticoid with reduced systemic side effects, such as prednisone, prednisolone, flunisolide, triamcinolone acetonide, beclomethasone dipropionate, budesonide, fluticasone propionate, and mometasone furoate.

The present invention still further relates to the combination of the compound of the invention together with an inhibitor of matrix metalloproteases (MMPs), i.e., the stromelysins, the collagenases, and the gelatinases, as well as aggrecanase; especially collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-12.

The present invention still further relates to the combination of the compound of the invention together with other modulators of chemokine receptor function such as CCR1, CCR2, CCR2A, CCR2B, CCR3, CCR4, CCR5, CCR6, CCR7, CCR8, CCR9, CCR10 and CCR11 (for the C—C family); CXCR1, CXCR3, CXCR4 and CXCR5 (for the C—X—C family) and CX₃CR1 for the C—X₃—C family.

The present invention still further relates to the combination of the compound of the invention together with antiviral agents such as Viracept, AZT, aciclovir and famciclovir, and antisepsis compounds such as Valant.

The present invention still further relates to the combination of the compound of the invention together with cardiovascular agents such as calcium channel blockers, lipid lowering agents such as statins, fibrates, beta-blockers, ACE inhibitors, Angiotensin-2 receptor antagonists and platelet aggregation inhibitors.

The present invention still further relates to the combination of the compound of the invention together with CNS agents such as antidepressants (such as sertraline), anti-Parkinsonian drugs (such as deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase), and anti-Alzheimer's drugs such as donepezil, tacrine, COX-2 inhibitors, propentofylline or metryfonate.

The present invention still further relates to the combination of the compound of the invention together with (i) tryptase inhibitors; (ii) platelet activating factor (PAF) antagonists; (iii) interleukin converting enzyme (ICE) inhibitors; (iv) IMPDH inhibitors; (v) adhesion molecule inhibitors including VLA-4 antagonists; (vi) cathepsins; (vii) MAP kinase inhibitors; (viii) glucose-6 phosphate dehydrogenase inhibitors; (ix) kinjn-B.sub1.- and B.sub2.-receptor antagonists; (x) anti-gout agents, e.g., colchicine; (xi) xanthine oxidase inhibitors, e.g., allopurinol; (xii) uricosuric agents, e.g., probenecid, sulfinpyrazone, and benzbromarone; (xiii) growth hormone secretagogues; (xiv) transforming growth factor (TGFP); (xv) platelet-derived growth factor (PDGF); (xvi) fibroblast growth factor, e.g., basic fibroblast growth factor (bFGF); (xvii) granulocyte macrophage colony stimulating factor (GM-CSF); (xviii) capsaicin cream; (xix) Tachykinin NK.sub1. and NK.sub3. receptor antagonists selected from the group consisting of NKP-608C; SB-233412 (talnetant); and D-4418; (xx) elastase inhibitors selected from the group consisting of UT-77 and ZD-0892; (xxi) TNFα converting enzyme inhibitors (TACE); (xxii) induced nitric oxide synthase inhibitors (iNOS) or (xxiii) chemoattractant receptor-homologous molecule expressed on TH2 cells, (CRTH2 antagonists).

The compound of the present invention may also be used in combination with osteoporosis agents such as roloxifene, droloxifene, lasofoxifene or fosomax and immunosuppressant agents such as FK-506, rapamycin, cyclosporine, azathioprine, and methotrexate;.

The compound of the invention may also be used in combination with existing therapeutic agents for the treatment of osteoarthritis. Suitable agents to be used in combination include standard non-steroidal anti-inflammatory agents (hereinafter NSAID's) such as piroxicam, diclofenac, propionic acids such as naproxen, flubiprofen, fenoprofen, ketoprofen and ibuprofen, fenamates such as mefenamic acid, indomethacin, sulindac, apazone, pyrazolones such as phenylbutazone, salicylates such as aspirin, COX-2 inhibitors such as celecoxib, valdecoxib, rofecoxib and etoricoxib, analgesics and intraarticular therapies such as corticosteroids and hyaluronic acids such as hyalgan and synvisc and P2X7 receptor antagonists.

The compound of the invention can also be used in combination with existing therapeutic agents for the treatment of cancer. Suitable agents to be used in combination include:
(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegafur, raltitrexed, methotrexate, cytosine arabinoside, hydroxyurea, gemcitabine and paclitaxel (Taxol®); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);
(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;
(iii) Agents which inhibit cancer cell invasion (for example metalloproteinase inhibitors like marimastat and inhibitors of urokinase plasminogen activator receptor function);
(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;
(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αvβ3 function and angiostatin);
(vi) vascular damaging agents such as Combretastatin A4 and compounds disclosed in International Patent Applications WO 99/02166, WO00/40529, WO 00/41669, WO01/92224, WO02/04434 and WO02/08213;
(vii) antisense therapies, for example those which are directed to the targets listed above, such as ISIS 2503, an anti-ras antisense;
(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and
(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

### BRIEF DESCRIPTION OF THE DRAWINGS

FIG. 1 shows the X-ray powder diffraction (“XRPD”) pattern for modification A.

FIG. 2 shows the XRPD pattern for modification B.

FIG. 3 shows the XRPD pattern for modification C.

FIG. 4 shows the XRPD pattern for modification D.

FIG. 5 shows the XRPD pattern for modification E.

FIG. 6 shows the XRPD pattern for modification F.

The invention will now be illustrated but not limited by reference to the following Specific Description, Examples, Biological Data and Reference Examples:

### SPECIFIC DESCRIPTION

The compound of formula (1) has at least one improved pharmacological property compared with any one of the known compounds identified below (see Tables 1 and 2).

The hepatic metabolic component of human clearance is predicted from scaled in vitro intrinsic clearance (CLᵢₙₜ) data from human hepatocytes (see Chem Biol Interact. 2007, 168(1), 2-15) and from the extent of human blood binding, primarily due to plasma protein binding. The well stirred model of the liver is a model for predicting blood clearance in the liver from intrinsic clearance (CLᵢₙₜ) determined using hepatocytes. (see Drug Metab Dispos. 2005, 33(9), 1304-11) The model is usually written as:

$${Cl}_{human} ( ml \text{/} min \text{/} kg ) = \frac{\frac{Q \cdot A \cdot B \cdot {CL}_{int} \cdot {fu}_{human}}{1000 \cdot ( B / P ) \cdot {fu}_{inc}}}{\frac{A \cdot B \cdot {CL}_{int} \cdot {fu}_{human}}{1000 \cdot ( B / P ) \cdot {fu}_{inc}} + Q}$$

where A is millions of hepatocytes per gram of liver, B is grams of liver per kilogram of body weight (the standard values of these parameters are A=120 and B=22. 1), fuₕᵤₘₐₙ is the human free fraction in plasma, fu_{inc} is the free fraction in the hepatocyte matrix and B/P is the blood to plasma concentration ratio in human blood.

It is clear from the above model that reducing in vitro human hepatocyte intrinsic clearance (CLᵢₙₜ) reduces human metabolic clearance (CL). Reducing metabolic clearance (CL) increases elimination half-life (t_{1/2}) and thus duration of action of the drug as can be seen by considering the following well known equation:

$${t}_{1 / 2} = \frac{{V}_{d} \times 0.693}{CL}$$

Elimination half-life (t_{1/2}) is the time taken to reach half plasma concentrations (in the phase associated the largest area of the plasma concentration-time profile) and V_{d} is the volume of distribution (see Clinical Pharmacokinetics, concepts and applications, 3^{rd} edition. 1995. by M Rowland and T. N. Tozer. Publisher Williams and Wilkins and see Current Drug Matabolism. 2006, 7(3), 251-64).

It follows from the above that lower clearances (CLᵢₙₜ) and (CL) will impact both the dose required to achieve therapeutic concentrations of drug and also the frequency of dosing. A lower (CL) means a lower dose of drug is required to achieve therapeutic concentrations.

In particular, comparison of compounds from WO 2004/011443 i.e. Examples 21 and 39-42 (see Table 1), with the compound of Formula (1) (see Table 2) shows that the compound of Formula (1) has both improved potency (pIC₅₀=8.2) and reduced hepatic intrinsic clearance (Clᵢₙₜ=2.1) as a measure of its hepatic metabolic stability.

Specifically, Example 21(pIC₅₀=5.6) (Table 1) from WO 2004/011443 exhibited a low hepatic intrinsic clearance value (Clᵢₙₜ=2.3) comparable with the compound of formula (1) (Clᵢₙₜ=2.1). However, this compound is significantly less potent than the compounds of Examples 39-42 (316-1000 fold) and the compound of Formula (1) (398 fold).

Structural modifications encompassed in some compounds of Examples 39-42 (Table 1) from WO 2004/011443 led to higher potencies (pIC₅₀=8.1-8.6) compared to the compound of Formula (1) (pIC₅₀=8.2). However, the compounds of Examples 39-42 are metabolically less stable as evidenced by their higher hepatic intrinsic clearances compared with the compound of Example 21 from WO-2004/022443 (2.2-7.4 fold) and the compound of Formula (1) (2.4-8.1 fold). Additionally, the compound of formula (1) exhibits a favourable free fraction in human plasma. Improved free fraction in human plasma is expected to result in an improved overall human whole blood potency in man.

TABLE 1
Structures and pharmacological profile of compounds disclosed in WO 2004/011443

| | Potency | | | | |
|---|---|---|---|---|---|
| | ligand- | Human hepatocyte | | | |
| Example | binding | Intrinsic-clearance | Rat oral bio- | Solubility | Human plasma |
| No. | assay | assay CLᵢₙₜ | availability | S | protein binding |
| (Structure) | pIC₅₀ | (μL/min/10⁶ cells) | F (%) | (mg/mL) | PPB (% free) |
| 21 | 5.6 | 2.3 | — | — | — |
| 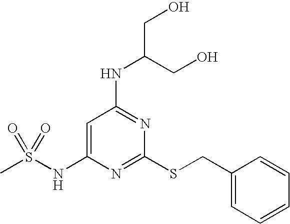 | | | | | |
| 39 | 8.4 | 5.1 | 44 | 342 | 1.0 |
| 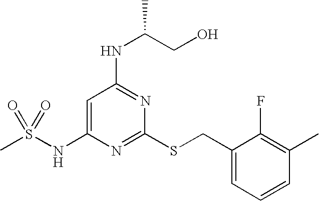 | | | | | |
| 40 | 8.6 | 9 | — | — | <0.2 |
| 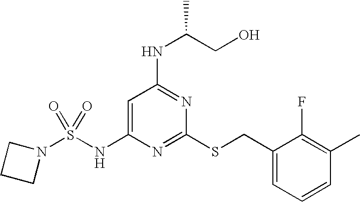 | | | | | |
| 41 | 8.5 | 12 | — | 372 | 0.6 |
| 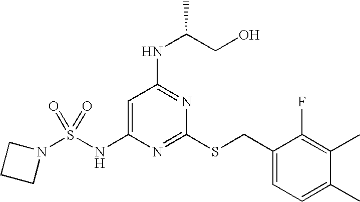 | | | | | |
| 42 | 8.1 | 17 | — | — | <0.2 |
| 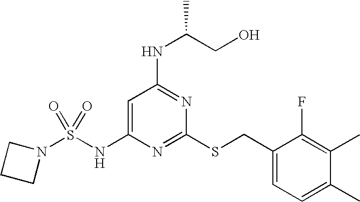 | | | | | |

— indicates data not determined

TABLE 2
Structure and pharmacological profile of compound of Formula (1)

| | | | | | |
|---|---|---|---|---|---|
| | Potency | | | | |
| | ligand- | Human hepatocyte | | | |
| Example | binding | Intrinsic-clearance | Rat oral bio- | Solubility | Human plasma |
| No. | assay | assay CLᵢₙₜ | availability | S | protein binding |
| (Structure) | pIC₅₀ | (μL/min/10⁶ cells) | F (%) | (mg/mL) | PPB (% free) |

| | | | | | |
|---|---|---|---|---|---|
| 1 | 8.2 | 2.1 | 49 | 317 | 1.9 |
| 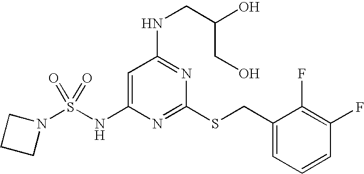 | | | | | |

The invention will now be illustrated by the following non-limiting Examples in which, unless stated otherwise:
(i) when given Nuclear Magnetic Resonance (NMR) spectra were measured on a Varian Unity Inova 300 or 400 MHz spectrometer. ¹H NMR data is quoted in the form of delta values for major diagnostic protons, given in parts per million (ppm) relative to tetramethylsilane (TMS) as an internal standard.
(ii) Mass Spectrometry (MS) spectra were measured on a Finnigan Mat SSQ7000 or Micromass Platform spectrometer.
(iii) the title and sub-titled compounds of the Examples and methods were named using the FUPAC ACD Name program (version 8.0) from Advanced Chemical Development Inc, Canada.
(iv) Normal phase column chromatography and normal phase HPLC was conducted using a silica column. Reverse phase High Pressure Liquid Chromatography (HPLC) purification was performed using either a Waters Micromass LCZ with a Waters 600 pump controller, Waters 2487 detector and Gilson FC024 fraction collector or a Waters Delta Prep 4000 or a Gilson Auto Purification System, using a Symmetry, NovaPak or Ex-Terra reverse phase silica column.
(v) Optical rotations were measured using a AA-1000 Polarimeter. [α]_{D} were measured at a temperature of 20° C. and at the wavelenghth of the Sodium D line, 589.3 nm
(vi) The X-ray powder diffraction (XRPD) analysis shown in FIGS. 1-6 was performed using a PANalytical CubiX PRO machine. The data was collected on the PANalytical CubiX PRO machine in θ-2θ configuration over the scan range 2° to 40° 2θ with 100-second exposure per 0.02° increment. The X-rays were generated by a copper long-fine focus tube operated at 45 kV and 40 mA. The wavelength of the copper X-rays was 1.5418 Å. The Data was collected on zero background holders on which˜2 mg of the compound was placed. The holder was made from a single crystal of silicon, which had been cut along a non-diffracting plane and then polished on an optically flat finish. The X-rays incident upon this surface were negated by Bragg extinction. All peaks stated are accurate to ±0.1 θ.
(vii) The following abbreviations are used:
   Xphos 2-dicyclohexyl-phosphino-2′,4′,6′-tri-isopropyl,1,1′-biphenyl
   AcOH acetic acid
   CHCl₃ chloroform
   DCM dichloromethane
   DMF N,N-dimethylformamide
   DMSO dimethylsulfoxide
   Et₂O diethyl ether
   EtOAc ethyl acetate
   MgSO₄ magnesium sulfate
   NMP 1-methylpyrrolidin-2-one
   THF tetrahydrofuran
   H₂O water
   NH₃ ammonia
   TFA trifluoroacetic acid
   MeOH methanol
   EtOH ethanol

### EXAMPLE 1

### N-(2-[(2,3-difluorobenzyl)thio]-6-{[(1R,2R)-2,3-dihydroxy-1-methylpropyl]amino}pyrimidin-4-yl)azetidine-1-sulfonamide

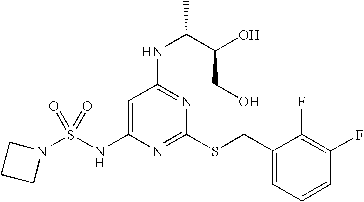 [0147] i) 1-[(4S)-2,2-dimethyl-1,3-dioxolan-4-yl]ethanone

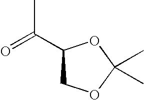

Citric acid (70 g, 0.37 mol) in water (67 mL) was added to a stirred solution of (S)-potassium 2,2-dimethyl-1,3-dioxolane-4-carboxylate (J. Med. Chem. 1991, 34, (1), 392-397), (75 g, 0.41 mol) in water (89 mL) and ethyl acetate (600 mL). The organic solution was separated and the aqueous solution extracted with ethyl acetate (3×300 mL). The combined organic extracts were dried (MgSO₄), filtered, concentrated in vacuo and then dried under high vacuum at room temperature to give a clear oil (59 g, 0.41 mol). The free acid ((4S)-2,2-dimethyl-1,3-dioxolane-4-carboxylic acid) was dissolved in dry diethyl ether (800 mL) with stirring and cooled to 0° C. under a nitrogen atmosphere. Methyl magnesium bromide (3M in diethyl ether, 200 mL, 0.60 moles) was added dropwise. A further quantity of dry diethyl ether (300 mL) was then added, followed by an additional quantity of methyl magnesium bromide (3M in diethyl ether, 97 mL, 0.29 mol). The addition was completed over 75 minutes. The reaction mixture was stirred at 0° C. for a further 30 minutes, was then allowed to warm to room temperature and was stirred for an additional 18 hours. Ethyl acetate (91 mL) was added dropwise over 5 minutes during which period the temperature rose from 21 to 25° C., and the mixture was stirred for 15 minutes. The reaction mixture was poured batchwise into aqueous ammonium chloride (230 g in 730 mL) pre-cooled in an ice bath to 5° C., during which time the temperature rose to 10° C. The organic phase was separated and the aqueous phase was extracted with diethyl ether (4×600 mL). The combined organic fractions were dried (MgSO₄), and concentrated in vacuo (bath temp<20° C.) to give the product as a pale yellow oil (27 g, 46%).

¹H NMR (400 MHz, CDCl₃): δ 4.41 (t, 1H), 4.20 (t, 1H), 4.00 (dd, 1H), 2.26 (s, 3H), 1.49 (s, 3H), 1.40 (s, 3H).
ii) (1R)-1-[(4R)-2,2-dimethyl-1,3-dioxolan-4-yl]-N-[(1R)-1-phenylethyl]ethanamine

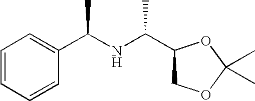

(R)-(α)-Methylbenzylamine (29.6 g, 31 mL, 0.24 mol) was added dropwise over 2 minutes to a stirred solution of the product of step i) (1-[(4S)-2,2-dimethyl-1,3-dioxolan-4-yl]ethanone) (27.1 g, 0.19 mol) in dry acetonitrile (430 mL) under a nitrogen atmosphere. The reaction mixture was cooled in a water bath as acetic acid (14.6 g, 13.9 mL, 0.24 mol) was added dropwise over 10 minutes. During this period the temperature was maintained between 20-23° C. After stirring for a further 10 minutes, sodium triacetoxyborohydride (99.7 g, 0.47 mol) was added batchwise over 1 hour, maintaining the temperature between 24 and 26° C. The resulting mixture was stirred at room temperature for 72 hours (over the weekend). The mixture was poured onto aqueous sodium bicarbonate and solid sodium bicarbonate was added until the effervescence ceased (pH 7-8). The organic solution was separated and the aqueous phase extracted with diethyl ether (2×500 mL). The combined organic extracts were washed with aqueous sodium chloride (300 mL), dried (MgSO₄) filtered and concentrated in vacuo to leave a two phase oil (clear/yellow) (43.5 g). Isohexane was added and the viscous lower layer was separated. The isohexane extract was then concentrated in vacuo to give the crude product as a pale yellow oil (43 g, 92%).

The above reaction was repeated twice more using 10.3 g and 33.6 g of (R)-(α)-methylbenzylamine with 9.4 g and 30.8 g of 1-[(4S)-2,2-dimethyl-1,3-dioxolan-4-yl]ethanone respectively to give 14.7 g and 43 g of crude product respectively. The combined crude products (100.7 g) were purified as follows:

The diastereomeric product mixture was purified in batches (approx. 22.5 g each run) by chromatography on silica (Biotage, EtOAc:isohexane:triethylamine 20:80:0.5). Appropriate fractions containing the desired product (top spot) were combined into two separate batches (Fraction 1: 32.9 g, and Fraction 2: 19.5 g) and rechromatographed separately (Fraction 1 in 2 batches, Fraction 2 in one batch) to give the subtitle compound as a pale yellow oil (39.2 g, 33%).
¹H NMR (300 MHz, CDCl₃): δ 7.31 (m, 4H), 7.23 (m, 1H), 4.01 (m, 2H), 3.84 (m, 2H), 2.73 (m, 1H), 1.43 (s, 3H), 1.36 (s, 3H), 1.31 (d, 3H), 0.95 (d, 3H).
GC MS Purity 100%
MS: APCI(+ve) 105 (base peak), 234 (M−15), 250[M+H]⁺
HPLC MS Purity 97.5%; (No impurity>0.8%)
[α]_{D}+33.17@589 nm, c=8.35 mg/ml MeOH.
Chiral HPLC Purity 100% @220 nm. (Chirobiotic V column 4.6×100 mm eluting with 6.7:3.3:90, 0.1% AcOH in MeOH:0.1% TEA in MeOH:MeOH, 1 mL/min, 20° C. over 15 min)
iii) tert-butyl {(1R)-1-[(4R)-2,2-dimethyl-1,3-dioxolan-4-yl]ethyl}carbamate

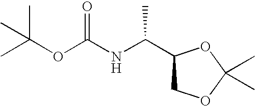

A mixture of the product of step ii) ((1R)-1-[(4R)-2,2-dimethyl-1,3-dioxolan-4-yl]-N-[(1R)-1-phenylethyl]ethanamine) (18.9 g, 76 mmol), di-tert-butyl dicarbonate (16.9 g, 76 mmol) and 20% palladium(II) hydroxide on carbon (0.92 g) in ethanol (270 mL) was hydrogenated at 4 atmosphere pressure hydrogen at room temperature with stirring over 72 hours (over the weekend). The reaction mixture was filtered through Hyflo and the solvent evaporated to give the subtitle compound as a colourless crystalline solid (18.7 g, 100%)
¹H NMR (400 MHz, CDCl₃): δ 4.56 (bs, 1H), 4.02 (t+bs, 2H), 3.76 (q+bs, 2H), 1.44 (s, 9H), 1.43 (s, 3H), 1.34 (s, 3H), 1.15 (d, 3H).
GC MS Purity 100%
MS: APCI(+ve) 57 (base peak), 230 (M−15)
[α]_{D}+12.49@589 nm, c=9.6 mg/ml MeOH
iv) (2R,3R)-3-aminobutane-1,2-diol hydrochloride

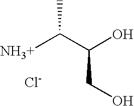

A solution of the product of step iii) (tert-butyl{(1R)-1-[(4R)-2,2-dimethyl-1,3-dioxolan-4-yl]ethyl}carbamate) (10 g, 41 mmol) in methanol (51 mL) was treated with 4M HCl in dioxane (51 mL) dropwise over 10 minutes with stirring, maintaining the temperature between 21° C. to 25° C. with a water bath, and the mixture was then stirred at room temperature for 18 h. The solvent was removed in vacuo, the residue was azeotroped twice with toluene and then dried under high vacuum to give the subtitle compound as a yellow viscous gum retaining some residual solvent (7.3 g).
¹H NMR (300 MHz, DMSO): δ 7.79 (bs, 3H), 3.67 (m, 1H), 3.42 (dd, 1H), 3.30 (m, 2H), 1.10 (d, 3H) v) (2R,3R)-3-({6-chloro-2-[(2,3-difluorobenzyl)thio]pyrimidin-4-yl}amino)butane-1,2-diol

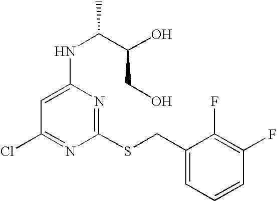

A mixture of the product of step iv) ((2R,3R)-3-aminobutane-1,2-diol hydrochloride) (3.3 g, (based on 75% by weight from NMR analysis), 2.5 g, 17 mmol), 4,6-dichloro-2-[(2,3-difluorobenzyl)thio]pyrimidine (WO-2004/011443) (5.0 g, 16 mmol) and sodium hydrogen carbonate (4.4 g, 53 mmol) in acetonitrile (80 mL) was heated at reflux with stirring under a nitrogen atmosphere for 18 h. The reaction mixture was cooled to room temperature, the solvent removed in vacuo and the residue partitioned between water and ethyl acetate. The organic phase was separated and washed with water and brine before being dried (MgSO₄), filtered and concentrated in vacuo to give a yellow oil (7.5 g). The oil was purified by chromatography on silica (Biotage, ethyl acetate:isohexane 8:2) to give the product as a white foam (5.7 g, 95%).
¹H NMR (300 MHz, DMSO): δ 7.70 (d, 1H), 7.32 (m, 2H), 7.15 (m, 1H), 6.32 (s, 1H), 4.83 (d, 1H), 4.59 (t, 1H), 4.37 (q, 2H), 4.21 (bm, 1H), 3.52 (m, 1H), 3.34 (m, 2H), 1.02 (d, 3H).
HPLC MS Purity 100%;
MS: APCI(+ve) 376/378 [M+H]⁺
vi) N-(2-[(2,3-difluorobenzyl)thio]-6-{[(1R,2R)-2,3-dihydroxy-1-methylpropyl]amino}pyrimidin-4-yl)azetidine-1-sulfonamide

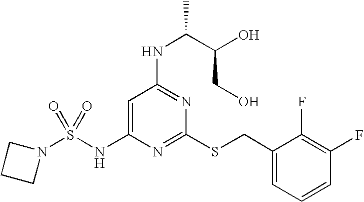

A mixture of the product of step v) ((2R,3R)-3-({6-chloro-2-[(2,3-difluorobenzyl)thio]pyrimidin-4-yl}amino)butane-1,2-diol) (5.3 g, 14 mmol), azetidine-1-sulfonamide (WO-2004/011443) (2.7 g, 19 mmol), palladium(II) tris(dibenzylideneacetone) dipalladium (0) (0.82 g), XPhos (0.82 g) and cesium carbonate (6.4 g, 20 mmol) in dry dioxane (85 mL) was heated at 105° C. for 90 minutes with stirring under a nitrogen atmosphere. The mixture was allowed to cool to room temperature, acetic acid (13 mL) was added and the solvent removed in vacuo. The residues were partitioned between water and ethyl acetate, and the organic fraction was separated, washed with water and brine, dried (MgSO₄), filtered and concentrated in vacuo to give a red foam (10.0 g). The product was purified twice by chromatography (SiO₂, EtOAc) to give a yellow foam which was suspended in DCM, refluxed for 10 minutes and then allowed to cool to room temperature overnight with stirring. The solid was filtered and dried under vacuum to give the title compound as a colourless solid (4.2 g, 63%) assigned as crystalline form modification A.
¹H NMR (400 MHz, DMSO): δ 10.49 (s, 1H), 7.35 (m, 2H), 7.14 (m, 1H), 5.99 (s, 1H), 4.71 (s, 1H),4.53 (s, 1H), 4.39 (q, 2H), 4.17 (bs, 1H), 3.88 (t, 4H), 3.48 (m, 1H), 2.12 (m, 2H), 1.04 (d, 3H), 3.33 (m (partially obscured by HOD signal), 2H)
HPLC MS Purity 99.2%;
MS: APCI(+ve) 476 [M+H]⁺
Elemental Analysis: Found: C, 45.32; H, 4.86; N, 14.79; S, 13.47%.
Calc for: [C₁₈H₂₃N₅O₄S₂F₂]: C, 45.46; H, 4.87; N, 14.73; S, 13.48%.
m.p. 116-116.5° C.
[α]_{D}+28.3@589 nm, c=0.972 mg/ml MeOH
Chiral HPLC Purity 98.3%@220 nm. (Chiralcel OD column 4.6×250 mm eluting with 90:10, 0.1% TFA in isohexane: isopropanol, lmL/min, 40° C. over 90 min) The crystallinity of modification A was improved by slurrying the material (10.8 mg) in water (150 μl) at room temperature for one week. The solid was isolated from the slurry after a week and was analysed by XRPD. The XRPD pattern for modification A is shown in FIG. 1. Some of the characteristic peaks for modification A are listed in Table 3.

TABLE 3
Some characteristic peaks for modification A

| Pos. [°2Th.] | d-spacing [Å] |
|---|---|
| 6.7 | 13.1 |
| 8.8 | 10.0 |
| 11.6 | 7.6 |
| 13.5 | 6.5 |
| 17.5 | 5.1 |

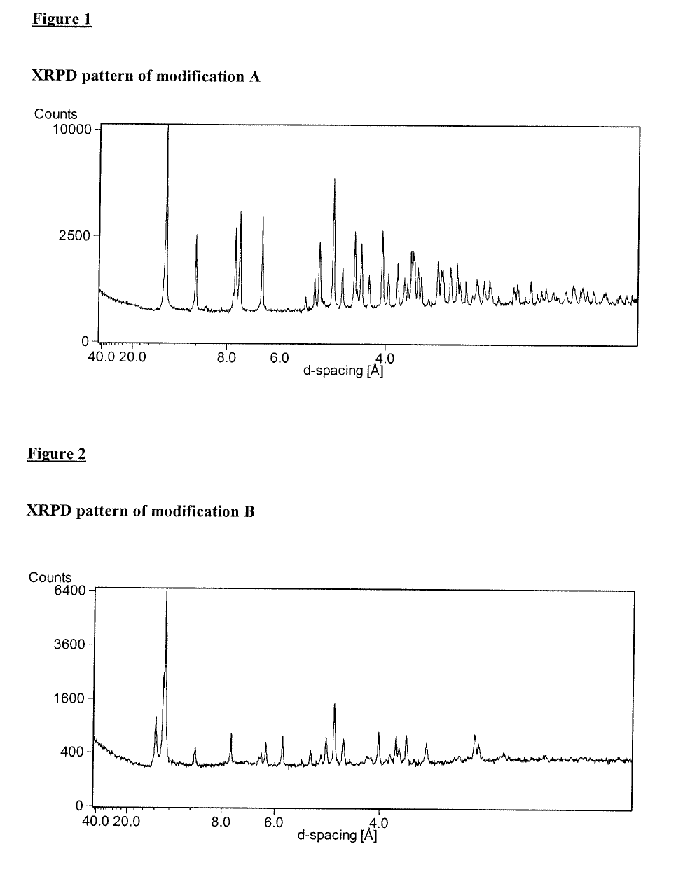

Modification B was prepared by slurrying modification A (8.9 mg) in cyclohexane (70 μl) at room temperature for one week. The solid was isolated from the slurry after a week and was analysed by XRPD. The XRPD pattern for modification B is shown in FIG. 2. Some of the characteristic peaks for modification B are listed in Table 4. Modification B was also produced by slurrying modification A in iso-propanol at room temperature and in hexane, cyclohexane, water or toluene at 70° C. all for one week.

TABLE 4
Some characteristic peaks for modification B

| Pos. [°2Th.] | d-spacing [Å] |
|---|---|
| 7.1 | 12.5 |
| 11.7 | 7.6 |
| 15.3 | 5.8 |
| 22.1 | 4.0 |

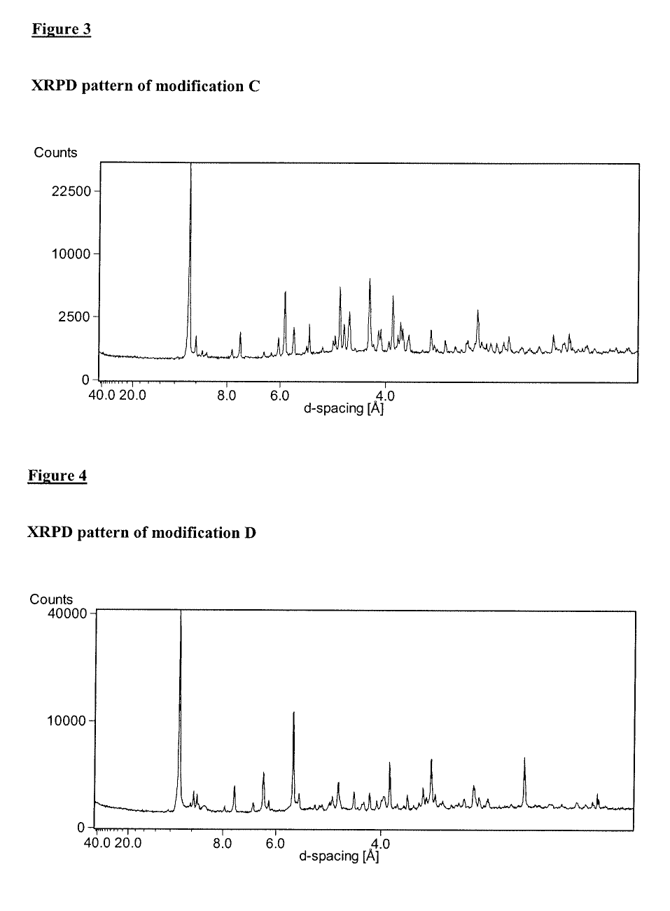

Modification C was prepared by slurrying modification A (9.6 mg) in dioxane (50 μl) at room temperature for one week. The solid was isolated from the slurry after a week and was analysed via XRPD. The XRPD pattern for modification C is shown in FIG. 3. Some of the characteristic peaks for modification C are listed in Table 5.

TABLE 5
Some characteristic peaks for modification C

| Pos. [°2Th.] | d-spacing [Å] |
|---|---|
| 8.4 | 10.5 |
| 14.7 | 6.0 |
| 15.1 | 5.9 |
| 15.7 | 5.6 |
| 16.8 | 5.3 |

Modification D was prepared by slurrying modification A (9.1 mg) in ethyl acetate (50 μl) at room temperature for one week. The solid was isolated from the slurry after a week and was analysed via XRPD. The XRPD pattern for modification D is shown FIG. 4. Some characteristic peaks for modification D are listed in Table 6. Modification D was also prepared by slurrying modification A in ethyl acetate at 70° C. for one week.

TABLE 6
Some characteristic peaks for modification D

| Pos. [°2Th.] | d-spacing [Å] |
|---|---|
| 8.0 | 11.1 |
| 9.0 | 9.9 |
| 9.2 | 9.6 |
| 11.9 | 7.5 |
| 13.9 | 6.4 |

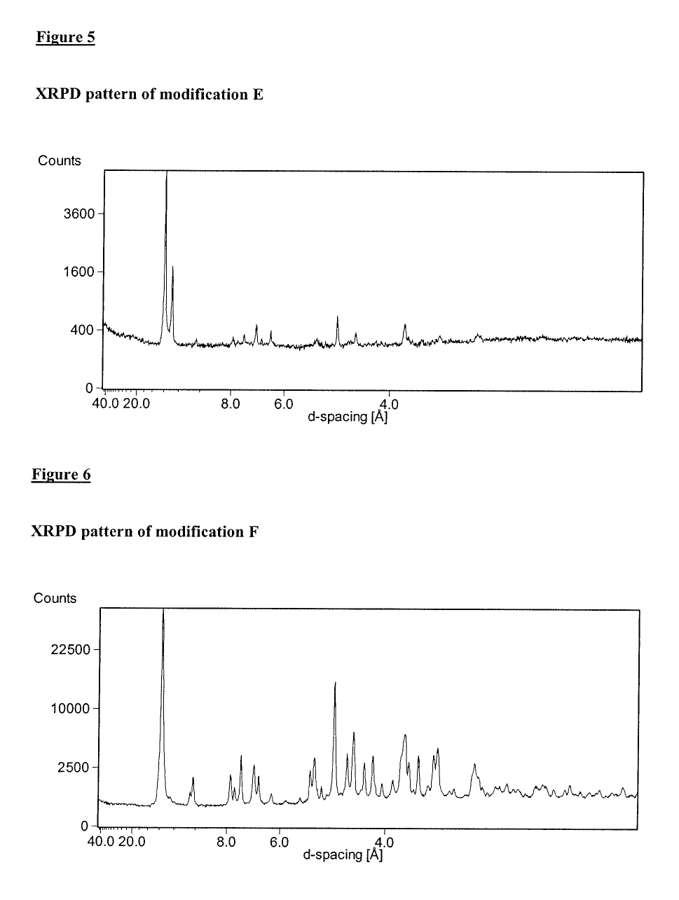

Modification E was prepared by slurrying modification A (6.8 mg) in hexane (100 μl) at room temperature for one week. The solid was isolated from the slurry after a week and was analysed via XRPD. The XRPD pattern for modification E is shown in FIG. 5. Some of the characteristic peaks for modification E are listed in Table 7.

TABLE 7
Some characteristic peaks for modification E

| Pos. [°2Th.] | d-spacing [Å] |
|---|---|
| 11.2 | 7.9 |
| 12.8 | 6.9 |
| 18.5 | 4.8 |
| 19.8 | 4.5 |

Modification F was prepared by slurrying modification A (9.1 mg) in diethyl ether (70 μl) at room temperature for one week. The solid was isolated from the slurry after a week and was analysed by XRPD. The XRPD pattern for modification F is shown in FIG. 6 below. Some of the characteristic peaks for modification F are listed in Table 8.

TABLE 8
Some characteristic peaks for modification F

| Pos. [°2Th.] | d-spacing [Å] |
|---|---|
| 8.7 | 10.2 |
| 13.0 | 6.8 |
| 13.3 | 6.7 |
| 16.9 | 5.3 |
| 19.9 | 4.5 |

### EXAMPLE 2

### Alternative Preparation of the Compound of Example 1

a) (1R)-1-[(4R)-2,2-dimethyl-1,3-dioxolan-4-yl]ethanamine

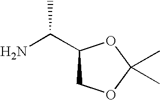

To the product of Example 1 step ii) ((1R)-1-[(4R)-2,2-dimethyl-1,3-dioxolan-4-yl]-N-[(1R)-1-phenylethyl]ethanamine) (2 g, 8.0 mmol) in ethanol (30 mL) was added palladium hydroxide (0.05 g, 20% Pd) and the mixture was hydrogenated with stirring at 5 bar at room temperature over 16 hours. Additional palladium hydroxide (0.2 g) was added and the mixture hydrogenated for a further 72 hours. The mixture was filtered through Hyflo and concentrated in vacuo to give the product as a clear oil (0.79 g, 67%).
¹H NMR (400 MHz, CDCl₃): δ 4.00 (t, 1H), 3.93 (mq, 1H), 3.81 (t, 1H), 3.06 (m, 1H), 1.43 (s, 3H), 1.36 (s, 3H), 1.08 (d, 3H).
GC MS Purity 100%
MS: APCI(+ve) 44 (base peak), 145 [M+H]⁺
b) 6-chloro-2-[(2,3-difluorobenzyl)thio]-N-{(1R)-1-[(4R)-2,2-dimethyl-1,3-dioxolan-4-yl]ethyl}pyrimidin-4-amine

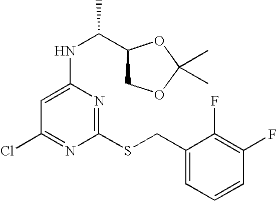

A mixture of the product of step a) ((1R)-1-[(4R)-2,2-dimethyl-1,3-dioxolan-4-yl]ethanamine) (0.40 g, 2.8 mmol), 4,6-dichloro-2-[(2,3-difluorobenzyl)thio]pyrimidine (WO-2004/011443) (0.77 g, 2.5 mmol) and sodium hydrogen carbonate (0.24 g, 2.8 mmol) in acetonitrile (12 mL) was heated at reflux with stirring under a nitrogen atmosphere for 18 h. The reaction mixture was cooled to room temperature, the solvent removed in vacuo and the residue partitioned between water and ethyl acetate. The organic phase was separated and washed with water and brine before being dried (MgSO₄), filtered and concentrated in vacuo to give a yellow oil (1.2 g). The oil was purified by chromatography on silica (Biotage, ethyl acetate:isohexane 2.5:7.5) to give the subtitle compound as a clear viscous oil (1.1 g, 95%).
¹H NMR (300 MHz, CDCl₃): δ 7.28 (m, 2H), 7.02 (m, 2H), 6.07 (s, 1H), 5.00 (bs, 1H), 4.42 (t, 2H), 4.05 (m, 2H), 3.76 (dd, 1H), 1.42 (s, 3H), 1.33 (s, 3H), 1.17 (d, 3H).
HPLC MS Purity 100%;
MS: APCI(+ve) 416/418 [M+H]⁺
c) N-[2-[(2,3-difluorobenzyl)thio]-6-({(1R)-1-[(4R)-2,2-dimethyl-1,3-dioxolan-4-yl]ethyl}amino)pyrimidin-4-yl]azetidine-1-sulfonamide

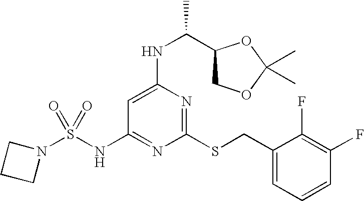

A mixture of the product of step b) (6-chloro-2-[(2,3-difluorobenzyl)thio]-N-{(1R)-1-[(4R)-2,2-dimethyl-1,3-dioxolan-4-yl]ethyl}pyrimidin-4-amine) (1.1 g, 25 mmol), azetidine-1-sulfonamide (WO-2004/011443) (0.51 g, 3.8 mmol), palladium(II) tris(dibenzylideneacetone) dipalladium (0) (0.15 g), XPhos (0.15 g) and cesium carbonate (1.2 g, 20 mmol) in dry dioxane (15 mL) was heated in a microwave in an open vessel at 100° C./300 W max for 12 minutes with stirring. The mixture was allowed to cool to room temperature, acetic acid (2.4 mL) was added and the solvent removed in vacuo. The residues were partitioned between water and ethyl acetate, and the organic fraction was separated, washed with water and brine, dried (MgSO₄), filtered and concentrated in vacuo to give a red gum (1.7 g). The product was purified twice by chromatography (SiO₂, EtOAc:isohexane 1:1 then EtOAc:isohexane 4:6) to give the product as a colourless foam (1.0 g, 75%).
¹H NMR (300 MHz, CDCl₃): δ 7.22 (m, 1H), 7.02 (m, 2H), 5.99 (s, 1H), 4.96 (bd, 1H), 4.35 (q, 2H), 4.15 (m, 2H), 3.98 (t, 4H), 3.78 (dd, 1H), 2.24 (m, 2H), 1.44 (s, 3H), 1.34 (s, 3H), 1.18 (d, 3H).
HPLC MS Purity 98.0%;
MS: APCI(+ve) 516 [M+H]⁺
d) N-(2-[(2,3-difluorobenzyl)thio]-6-{[(1R,2R)-2,3-dihydroxy-1-methylpropyl]amino}pyrimidin-4-yl)azetidine-1-sulfonamide

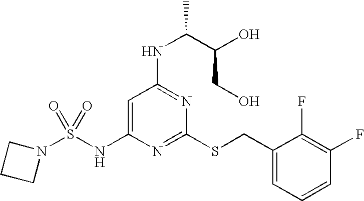

A mixture of the product of step c) (N-[2-[(2,3-difluorobenzyl)thio]-6-({(1R)-1-[(4R)-2,2-dimethyl-1,3-dioxolan-4-yl]ethyl}amino)pyrimidin-4-yl]azetidine-1-sulfonamide) (0.87 g, 1.7 mmol) and para-toluenesulfonic acid (0.85 g, 3.4 mmol) in methanol (19.5 mL) and water (5 drops) was heated at 60° C. for 20 hours. The solvent was evaporated and the residue taken up in ethyl acetate which was washed with water, dried (MgSO₄) and evaporated to give a pale yellow foam (0.74 g). Purification by chromatography (SiO₂, EtOAc:isohexane 9:1) gave a foam which was dried under high vacuum at 40° C. for 18 hours to give the title compound as a colourless solid (0.54 g, 67%)
¹H NMR (300 MHz, DMSO): δ 10.49 (s, 1H), 7.35 (m, 2H), 7.14 (m, 1H), 5.99 (s, 1H), 4.71 (s, 1H),4.53 (s, 1H), 4.39 (q, 2H), 4.17 (bs, 1H), 3.88 (t, 4H), 3.48 (m, 1H), 2.12 (m, 2H), 1.04 (d, 3H), 3.33 (m (partially obscured by HOD signal), 2H)
MS: APCI(+ve) 476 [M+H]⁺
Elemental Analysis: Found: C, 45.15; H, 4.79; N, 14.50; S, 13.36%.
Calc for: [C₁₈H₂₃N₅O₄S₂F₂]: C, 45.46; H, 4.87; N, 14.73; S, 13.48%.

### EXAMPLE 3

### Preparation of the Compound of Example 1 Repeated on Larger Scales Using the Route Outlined in Scheme 1 (Shown Below)

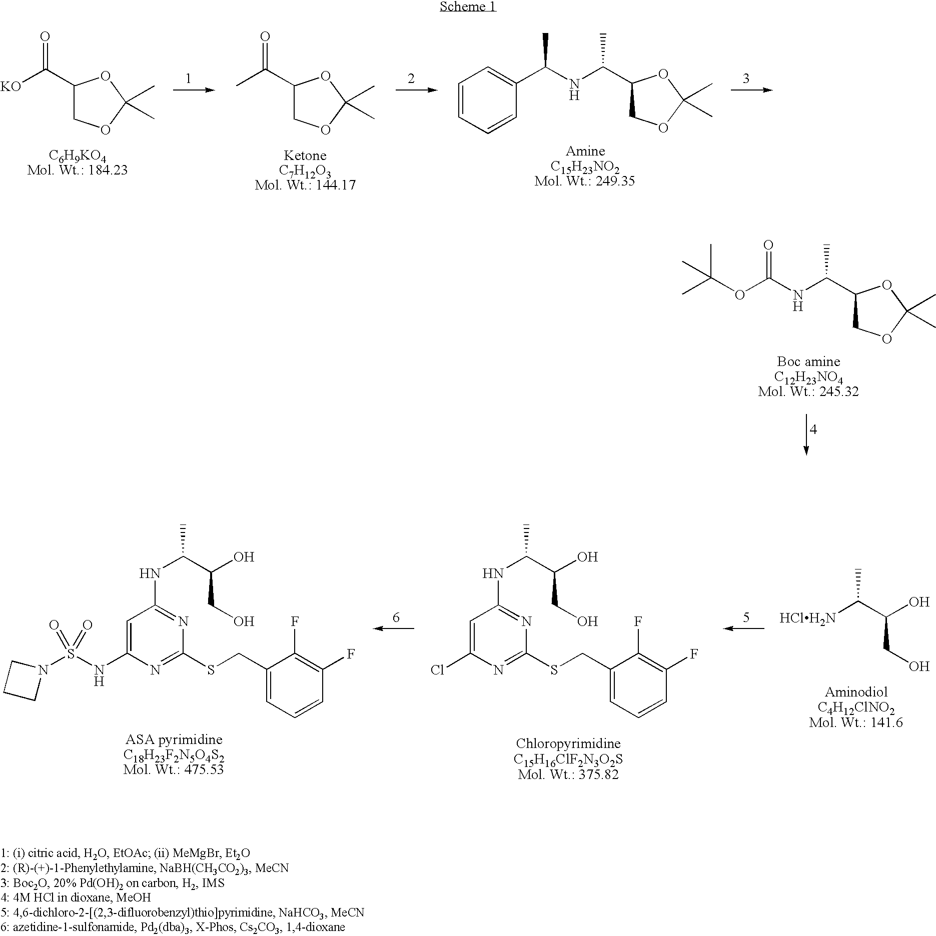

### Step 1

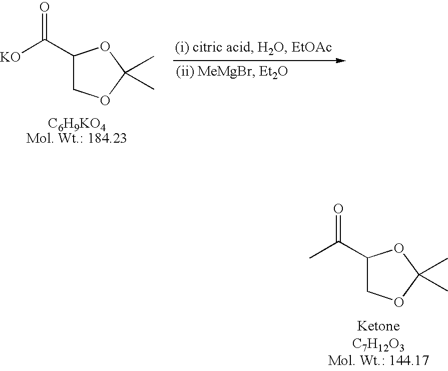

Citric acid (848 g, 4.41 mol) in water (800 ml) was added to a stirred solution of potassium 2,2-dimethyl-1,3-dioxolane-4-carboxylate (J. Med. Chem. 1991, 34, (1), 392-397), (900 g, 4.89 mol) in water (1062 ml) and ethyl acetate (7150 ml) then stirred for 15 minutes to give a colourless two phase solution. No exotherm was observed during the addition. The organic phase was separated and dried (MgSO₄). The aqueous layer was extracted with ethyl acetate (2×3500 ml) and the organics were dried (MgSO₄). The organic fractions were combined, concentrated in vacuo and dried under high vacuum at room temperature to give a clear oil (685.1 g, 4.66 mol). The oil was stored at −30° C. for 2 days with no effect on product quality by ¹H NMR analysis. The oil was dissolved in diethyl ether (13000 ml) and cooled to 5° C. under a nitrogen atmosphere. Methyl magnesium bromide (3.0M in diethyl ether, 3500 ml, 10.50 mol) was added to the reaction dropwise over a period of 90 minutes maintaining the reaction temperature between 0-10° C. Upon completion of the addition the mixture was stirred at 10° C. for 30 minutes then allowed to warm to room temperature with stirring overnight. Methyl acetate (75 ml, 0.94 mol) was added to the reaction mixture resulting in gas evolution and a slight exotherm. The reaction mixture was added to aqueous ammonium chloride (2750 g in 8700 ml) maintaining the temperature below 25° C. during the addition and stirred for 10 minutes. The organic phase was separated and the aqueous phase extracted with diethyl ether (3×7100 ml). The combined organic extracts were dried (MgSO₄) and concentrated in vacuo to give the ketone as a yellow oil.

| Experimental | Quantity of S.M. | Quantity of | Yield | Purity (%) by |
|---|---|---|---|---|
| repeats | (g) | Ketone (g) | (%) | ¹H NMR |
| 1 | 75 | 29.4 | 49.7 | >95% |
| 2 | 900 | 348.6 | 49.5 | >95% |
| 3 | 900 | 387.3 | 54.9 | ~90% |

### Step 2

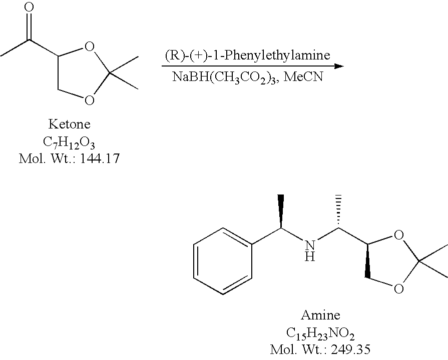

(R)-(+)-1-Phenylethylamine (715 g, 5.90 mol) was added dropwise over 55 minutes to a stirred solution of the ketone (700 g, 4.86 mol) in acetonitrile (11100 ml) under a nitrogen atmosphere. A small exotherm was observed during the addition. The reaction mixture was cooled to 10° C. and acetic acid (348 ml, 6.03 mol) was added dropwise over 45 minutes maintaining the temperature below 25° C. resulting in the formation of a white precipitate. After stirring for a further 10 minutes, sodium triacetoxyborohydride (2340 g, 11.04 mol) was added in portions over 1 hour maintaining the temperature below 25° C. and gas evolution was observed. The mixture was stirred at room temperature overnight. The reaction mixture was then added to water (11000 ml) with stirring under a nitrogen atmosphere (5 L/min flow rate) over 90 minutes. The addition resulted in a decrease in temperature and gas evolution. Sodium bicarbonate (1560 g, 18.57 mol) was added to the mixture in portions until the solution reached pH 7. The addition resulted in an exothenn and gas evolution. The organic phase was separated and the aqueous phase extracted with diethyl ether (2×10000 ml). The combined organic extracts were washed with aqueous sodium chloride (2760 g in 7000 ml), dried (MgSO₄) filtered and concentrated in vacuo to give a two phase oil (clear/yellow). Heptane (2000 ml) was added and the viscous lower layer separated. The heptane extract than was then concentrated in vacuo to give the crude product as a pale yellow oil (929.3 g, 76.7%). The diastereomeric product mixture was purified by chromatography on silica (ethyl acetate:heptane:triethylamine 20:80:0.5) in batches to give the product as a yellow oil. Amine isolated with lower diasteromeric purity was rechromatographed to give a second batch of product.

| Experimental | Quantity of | Quantity of Amine | Yield | de (%) by |
|---|---|---|---|---|
| repeats | Ketone (g) | (g) | (%) | chiral LC |
| 1 | 28.1 | 17.8 | 35.7 | 98.7% |
| 2 | 900 | 463.8 | 37.0 | >99% |

### Step 3

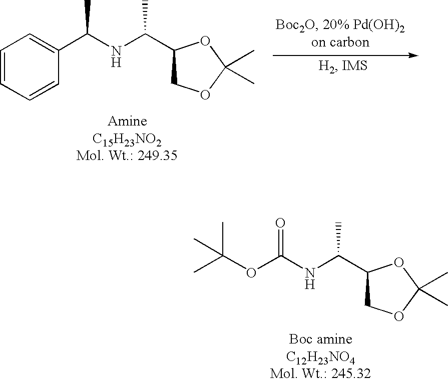

A mixture of the amine (236.1 g, 0.95 mol), di-tert-butyldicarbonate (208.0 g, 0.95 mol) and 20% palladium(II) hydroxide on carbon (11.5 g) in IMS (3375 ml) was hydrogenated at 4 bar pressure hydrogen at room temperature with stirring over 7 days. The reaction mixture was filtered through Hyflo and concentrated in vacuo to give a colourless crystalline solid.

| Experimental | Quantity of | Quantity of Boc | Yield | Purity (%) by |
|---|---|---|---|---|
| repeats | Amine (g) | amine (g) | (%) | ¹H NMR |
| 1 | 12.8 | 11.3 | 89.4 | >95% |
| 2 | 200.0 | 192.2 | 97.3 | >95% |
| 3 | 236.1 | 227.2 | 97.5 | >95% |

### Step 4

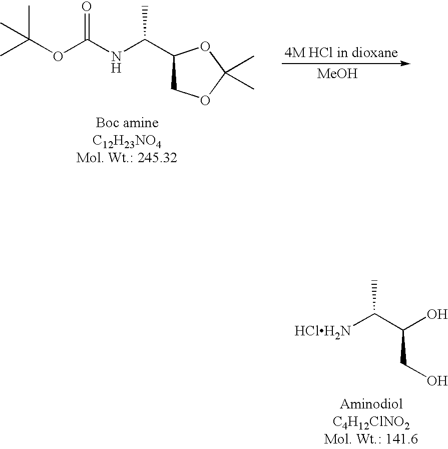

4M HCl in dioxane (1800 ml, 7.22 mol) was added dropwise to a cooled solution of the Boc amine (353.5 g, 1.44 mol) in methanol (1800 ml) under a nitrogen atmosphere. The temperature of the reaction ranged from 14 to 20° C. with a water bath present during the addition. The mixture was then stirred at room temperature for 18 hours. The solvent was removed in vacuo, the residue azeotroped twice with toluene (2×500 ml) and then dried is under high vacuum to give a brown viscous gum.

| Experimental | Quantity of Boc | Quantity of Aminodiol | Purity (%) |
|---|---|---|---|
| repeats | amine (g) | (g) | by ¹H NMR |
| 1 | 11.3 | 7.1 | ~75% |
| 2 | 50.0 | 36.8 | ~75% |
| 3 | 353.3 | 266.4 | ~75% |

### Step 5

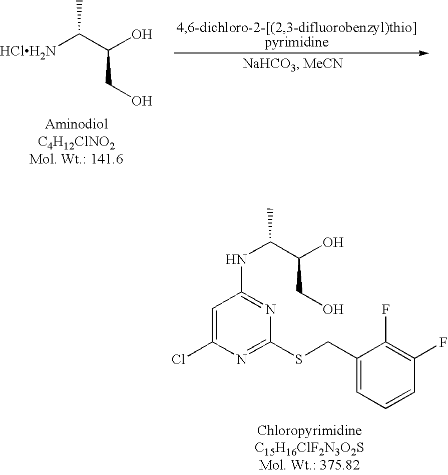

A mixture of the aminodiol (266.4 g, approx. 75% by weight, 199.8 g, 1.38 mol), 4,6-dichloro-2-[(2,3-difluorobenzyl)thio]pyrimidine (390.0 g, 1.27 mol) and sodium bicarbonate (361.0 g, 4.30 mol) in acetonitrile (6500 ml) was heated at reflux with stirring under a nitrogen atmosphere for 17 hours. During this time an off white suspension formed. The reaction mixture was cooled to room temperature, the solvent removed in vacuo and the residue partitioned between ethyl acetate (4000 ml) and water (4000 ml). The organic layer was separated and washed with water (2000 ml) and brine (2000 ml) before being dried (MgSO₄), filtered and concentrated in vacuo to give a dark yellow oil. The oil was purified by chromatography on silica (ethyl acetate:heptane 4:1) to give the chloropyrimidine as a yellow gum.

| | | Quantity of | | |
|---|---|---|---|---|
| Experimental | Quantity of | Chloropyrimidine | Yield | Purity (%) by |
| repeats | Aminodiol (g) | (g) | (%) | ¹H NMR |
| 1 | 36.8 | 54.7 | 74.6 | >90% |
| 2 | 266.4 | 347.0 | 66.8 | ~90% |

### Step 6

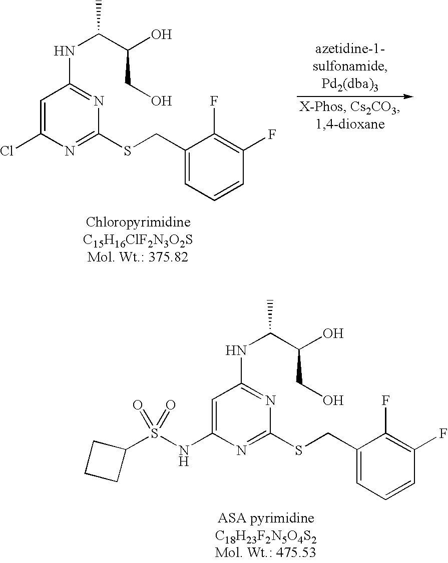

A mixture of the chloropyrimidine (382.1 g, 1.02 mol), azetidine-1-sulfonamide (200.0 g, 1.48 mol), di-Palladium-tris(dibenzylideneacetone) (56.1 g), X-Phos (56.5 g) and cesium carbonate (465.0 g, 1.43 mol) in 1,4-dioxane (6400 ml) was heated at 105° C. for 90 minutes under a nitrogen atmosphere with stirring. The reaction mixture was allowed to cool to room temperature and acetic acid (950 ml) was added to the mixture and stirred for 10 minutes. An exotherm was observed during the addition. The red solution had solvent removed in vacuo and the residues were partitioned between ethyl acetate (3500 ml) and water (3500 ml). The organic phase was separated, washed with water (2500 ml) and brine (2500 ml), dried (MgSO₄) and filtered. The resultant red solution was concentrated in vacuo to give a red foam. The product was purified by chromatography on silica (ethyl acetate:heptane 1:1 followed by ethyl acetate) to give a yellow foam. The yellow foam was dissolved in dichloromethane, refluxed for 10 minutes, resulting in formation of a pale yellow precipitate and allowed to cool to room temperature. The precipitate was filtered and then recrystallised (ethyl acetate:heptane), filtered and dried under vacuum at 60° C. to give the ASA pyrimidine as a colourless solid. The solid was further suspended in DCM (2 L) at room temperature for 5 days with stirring. The solid was filtered and dried under vacuum to give the title compound of Example 1 as a colourless solid.

| | | Quantity | | | |
|---|---|---|---|---|---|
| | | of ASA | | | ee (%) |
| | Quantity of | pyrimidine | | Purity | by |
| Experimental | Chloropyrimidine | (Example 1) | Yield | (%) by | chiral |
| repeats. | (g) | (g) | (%) | LCMS | LC |
| 1 | 20 | 14.8 | 58.6 | >98% | >99% |
| 2 | 382.1 | 270.5 | 56.0 | >98% | >99% |

### Biological Data

### Human Hepatic Intrinsic Clearance (CLᵢₙₜ) Assay

For the majority of drugs, a large component of their plasma clearance is contributed by hepatic metabolism. Intrinsic clearance (CLᵢₙₜ) is a measure of the potential of a compound to undergo metabolism and can be related to hepatic clearance in vivo from a consideration of plasma protein binding and liver blood flow. Therefore, CLᵢₙₜ may be used as an index of the relative metabolic stability of compounds within a project and compared with other external probe substrates. Furthermore, the measurement of CLᵢₙₜ in vitro within a research project, where hepatic metabolic clearance is known to be an issue, may be a useful means of understanding the different pharmacokinetic behaviour of the compounds in vivo.

### Test Description

This following description outlines a method for estimating intrinsic clearance (CLᵢₙₜ) from human hepatocyte incubations using suspension buffer containing no HSA (human serum albumin) and maintaining physiological conditions of pH 7.4.

In order for a skilled scientist to reproduce the operating characteristics of this test procedure, reference is made to specific suppliers and catalogue numbers for the reagents used at the time of initial validation and finalisation of the test procedure. This does not preclude substitution with suitable alternative reagents with either a documented comparable specification or following experimental confirmation that substitution does not significantly affect the operating characteristics of the assay.

Hepatocytes were prepared by a two-step in situ collagenase perfusion method of a portion of the human liver, suspended in protein free buffer (see below) and stored on ice, prior to incubation.

### Isolation of Human Hepatocytes by In Situ Collagenase Perfusion

This method is based on the procedure of Seglen (Preparation of rat liver cells. I. Effect of Ca²⁺ on enzymatic dispersion of isolated, perfused liver. Exptl. Cell Res., 1972, 74, p450 and preparation of isolated rat liver cells. Methods Cell Biol., 1976, 13, p 29) which itself was developed from the one step procedure of Berry and Friend (High-yield preparation of isolated rat liver parenchymal cells. J. Cell Biol., 1969, 43, p 506).

We now disclose the preparation of a protein free cell suspension.

### Chemicals and Reagents

5% Hydrogen peroxide: 60% (w/v) hydrogen peroxide (Fisher Scientific) diluted with Milli-Q water.

Liver perfusion medium: Supplied ready-to-use by Gibson Life Technologies (Cat no. 17701).

Liver digestion medium: Supplied ready-to-use by Gibson Life Technologies (Cat no. 17703).

Suspension medium: 2.34 g Na HEPES, 2.0 g HSA fraction V, 0.4 g D-fructose, DMEM (1 L powder equivalent, Sigma; w/1 g.1⁻¹ glucose, w/Na pyruvate, w/o NaHCO₃, w/o phenol red), made up to 1 L with Milli-Q water, pH to 7.4 with 1 M HCl. (Protein free suspension buffer is made omitting the 2.0 g HSA fraction V)

### Hepatocyte Isolation

The capsule of a liver which has been perfused with digestion medium was cut open and the cells gently teased out into the medium. The cells were then passed through a mesh (approximately 250 μM) into a beaker containing 50 ml suspension medium. The mesh was rinsed through into the beaker with further suspension buffer to a final volume of 100 ml. The suspension was divided between two plastic 50 mL centrifuge tubes (pre-cooled on ice) and centrifuged at 50×g for 2 min at 4° C. The supernatants were decanted and the pellets re-suspended in protein free suspension buffer to the original volume. The centrifugation step was repeated and each pellet re-suspended in approximately 10 ml protein free suspension buffer. The suspensions were combined and the volume made up to 50 mL with protein free suspension buffer.

### Estimation of Hepatocyte Yield and Viability

An aliquot of cell suspension (0.2 mL) was diluted with 0.2 ml protein free suspension buffer. To the diluted cells was added 0.2 mL trypan blue solution (0.4% w/v) followed by gentle mixing. After 1 min, a pasteur pipette was used to withdraw a sample and fill an Improved Neubauer Counting Chamber by capillary action. The cells were then counted (central square only) using an inverted microscope, viable cells being able to exclude the dye and non-viable cells being stained. The percentage of viable cells in the preparation was calculated thus:

$$\frac{Viable cell count}{Total cell count} \times \frac{100}{1} = \% viability$$

The concentration of viable cells was calculated:

Viable cells ml⁻¹=Viable cell count×10⁴×3×50

The counting procedure was performed in duplicate.

The cell suspension was diluted with an appropriate volume of protein free suspension buffer to give the required concentration of viable cells and stored on ice for up to 1 h prior to use.

### Removal of Protein

Fresh human hepatocytes are generally received in suspension buffer containing HSA. The procedure below describes the removal of the protein. Cryopreserved cells may simply be prepared using suspension buffer without protein.

Protein free suspension buffer was prepared in an analogous manner to the with protein suspension buffer, simply omitting the HSA. The cell suspension was re-centrifuged at 50×g, as described above and the supernatant discarded. This was then replaced with an appropriate volume of protein free suspension buffer. This process was repeated a second time to remove any remaining trace of protein, ensuring that the final re-suspension of the cells gives a concentration double that of the required incubation concentration.

### Test Procedure

The test compound to be incubated was added from a concentrated stock solution of 0.1 mM in DMSO (1% v/v final solvent concentration) to an appropriate volume (0.5 mL) of protein free suspension buffer in a suitable vial. An appropriate volume of cells (0.5 mL) at a concentration of 2×10⁶ cells·mL⁻¹ (twice the final incubation cell concentration, viability>85% by trypan blue exclusion) is placed in a separate vial and both vials are pre-incubated in a water bath at 37° C.

After 5 min pre-incubation an appropriate volume of the buffer and compounds were added to the cells in order to give a final cell concentration of 1×10⁶ cells·mL⁻¹ and the reactions allowed to proceed.

At appropriate time points (e.g. 5, 10, 20, 30, 60, 90 and 120 min), aliquots (50 μl) were taken out of the incubation mix and added to 2 volumes of a ice-cold solvent methanol to terminate the reactions and denature the hepatocytes. Control incubations were also conducted in which cells or compound were omitted. Once the incubations have been quenched, the samples were shaken for 5 min, stored at −20° C. or below for 2 h to aid protein precipitation and then centrifuged for 15 min at 3000 rpm and 4° C. The supernatants were transferred to HPLC vials and analysed by HPLC-MS using the following method as a suitable starting point:
Solvents: A: 0.1 % formic acid in methanol and B: 0.1 % formic acid in water (v/v)
Column: Waters Xterra C₁₈20×3.9 mm, 3.5 μm

Flow rate 1.5 ml.min⁻¹

Gradient: 0% B for 0.3 minutes, 0% to 100% B over 0.7 minutes, held at 100% B for 0.2 minutes, 100% to 0% B over 0.01 minutes.

### Data Analysis and Calculation Methods

The resultant peak areas of the incubated compounds are taken into an Excel spreadsheet and a plot of ln[residual concentration] versus time was produced. The treatment of the data is then akin to a one-compartment, pharmacokinetic model As dose/C₀ gives a term for the volume of the incubation (expressed in ml 10⁶ cells⁻¹) and the elimination rate constant k=0.693/t_{1/2}, an equation expressing Clᵢₙₜ in terms of t_{1/2} can be derived as given in Equation 1:

$$CL = \frac{Volume \times 0.693`}{{t}_{1 / 2}} \qquad Equation 1$$

The t_{1/2} and CLᵢₙₜ of the loss of the parent compound from the incubation was then determined.

Potency (pIC₅₀)—Ligand Binding Assay

The potency of antagonists at the human CXCR2 receptor was determined in vitro by quantifying their ability to inhibit specific binding of the CXCR2 radioligand, [¹²⁵I]interleukin-8 (IL-8), from membranes of HEK293 cells transfected with the human recombinant CXCR2 receptor.

### Experimental Procedure

### Materials

Commercially sourced materials were obtained as follows: U-bottomed 96-well plates (3799) and 225 cm² vented cap culture flasks (3001) from Costar, Corning, Kent, UK. Multiscreen filter plates (0.45 μm; MAHV N45 50), vacuum manifold and pump (XF54 230 50) from Millipore, Watford, UK. N-[2-hydroxyethyl]piperazine-N′-[2ethanesulphonic acid] (HEPES; H-3375), ethylene diamine-tetraacetic acid (EDTA; E1644), magnesium chloride (M-9272), gelatin (G9382), dithiothreitol (DTT; D06052), sodium chloride (S3160/63), sodium hydroxide (B6506), bacitracin (B0125), inactivated foetal calf serum (FCS; CR0848) and DMSO Fluka Chemika (41648) from Sigma, Poole, UK. MicroScint-O (6013611) Packard BioScience, Pangboume, UK. Complete protease inhibitor cocktail tablets (1836145) from Boehringer s Mannheim, GmbH, Germany. Human recombinant [¹²⁵I]IL-8 74 TBq/mmol, 0.712 MBq/ml (IM249) from Amersham, Horsham UK. All other tissue culture reagents were purchased from Invitrogen, Paisley, Scotland, UK. All other chemical reagents were analytical grade from Fisher Scientific, Loughborough, UK

### Solutions

HEPES-buffered salt solution pH 7.4 containing HEPES (10 mM), potassium chloride (2.7 mM), sodium chloride (137 mM), potassium hydrogen phosphate (0.4 mM), calcium chloride (1.8 mM), magnesium chloride (1 mM), gelatin (0.1% (w/v)) and bacitracin (100 μg/ml).

HEPES-buffered Tyrode's solution pH 7.4 containing HEPES (10 mM), potassium chloride (2.7 mM), sodium chloride (137 mM), potassium hydrogen phosphate (0.4 mM), glucose (11 mM).

Hypotonic buffer: 3:1 mix of water: HEPES-buffered Tyrode's solution.

### Cell Culture and Membrane Preparation

HEK293 cells were transfected with human CXCR2 (EMBL L19593) cDNA, previously cloned into the eukaryotic expression vector RcCMV. Cloned cell-lines were generated from stably-transfected geneticin-resistant populations. Cells were routinely grown to approximately 80% confluence in DMEM medium containing 10% (v/v) foetal calf serum and glutamine (2 mM) in a humidified incubator at 37° C., 5% CO₂. Cells were harvested from flasks using Accutase™ at 37° C. for 3 to 5 minutes and resuspended on ice in hypotonic buffer at a density of 2×10⁷ cells/mL. Membranes were prepared on ice by homogenisaton using a polytron tissue homogenizer set at 22000 rpm. The membrane fraction was purified by sucrose gradient centrifugation where homogenised cells were layered onto 41% (w/v) sucrose solution then centrifuged at 140000 g for 1 hour at 4° C. The membrane fraction was harvested at the interface, diluted 4-fold with HEPES-buffered Tyrode's solution and centrifuged at 100000 g for 20 minutes at 4° C. The membrane pellet was re-suspended at 1×10⁸ cell equivalents/mL in HEPES-buffered Tyrode's solution and subsequently stored in aliquots at −80° C. All buffers used for membrane preparation and storage were made in the presence of 1 mM DTT and Complete Protease Inhibitor™ cocktail tablets, made up to manufacturers instructions.

### Assay Protocol

Assays were performed in HEPES-buffered salt solution in 96-well plates. [¹²⁵I]IL-8 was used at a final concentration of 0.06 nM, pre-diluted from a 9.6 nM stock. The final DMSO concentration in the assay was 1% (v/v). Test compounds were prepared by serial dilution in DMSO followed by a ten-fold dilution into HEPES-buffered salt solution to give a working solution containing compound and 10% DMSO. The control for total binding (B0) of [¹²⁵I]IL-8 was determined in the absence of compound. The control for non-specific binding (NSB) was determined by measuring [¹²⁵I]IL-8 binding in the presence of (1R)-5-[[(3 -chloro-2-fluorophenyl)methyl]thio]-7-[[2-hydroxy-1-methylethyl]amino]thiazolo[4,5-d]pyrimidin-2(3H)-one dihydrate, sodium salt at 1 μM final concentration. Frozen aliquots of membranes were defrosted and diluted to a concentration previously determined to give approximately 10% binding of total radiolabel added, typically about 1×10⁶ cell equivalents/mL. The assay components were added to each well as follows; one-tenth volume test compounds or controls in buffer containing 10% DMSO, one-tenth volume radiolabel, eight-tenths volume diluted membranes. The plates were sealed and incubated for 2 hours at room temperature. Following incubation, the assay mixture was filtered then washed with two volumes of cold HEPES-buffered salt solution using a Millipore vacuum manifold. The filtration plate was allowed to air dry then either the individual filters were punched out into polypropylene test tubes and the radioactivity measured by direct gamma counting using a Cobra II Gamma counter (Packard BioScience) for 1 minute per sample or alternatively, the whole filtration plate was placed in a carrier plate and 50 μL of MicroScint-O added to each well. 96-well plate scintillation counting was performed using a TopCount instrument (Packard BioScience) for 1 minute per sample well.

### Data Analysis

Specific binding of [¹²⁵I]IL-8 was calculated by subtracting the mean of the control NSB values determined in each assay plate. Data was transformed into concentration-response plots and expressed as a percent relative to total specifically bound [¹²⁵I]IL-8 (B0-NSB). The IC₅₀ was defined as molar concentration of compound required to give 50% inhibition of specifically bound [¹²⁵I]IL-8. The IC₅₀ values were transformed into the reciprocal logarithm (pIC₅₀) for calculation of descriptive statistics (mean±SEM). The pIC₅₀ values approximated to the binding affinity (pKi) since the concentration of [¹²⁵I]IL-8 used (0.06 nM) was below the Kd (equilibrium dissociation constant) determined for IL-8 (1.2 nM).

The compound of formula (1) was found to have a pIC₅₀ value of >8

### Measurement of Plasma Protein Binding (PPB)

The extent of binding of a drug to plasma proteins is a crucial factor in determining its in vivo potency and pharmacokinetics. The method used for determining the extent of plasma protein binding involves equilibrium dialysis of the compound between plasma and buffer at 37° C. The concentrations of compound in the plasma and buffer are then determined using high pressure liquid chromatography (HPLC) with mass spectroscopy (MS) detection. The dialysis method involves the use of mixtures of up to 10 compounds simultaneously. It has been shown that at the concentrations used in the assay, there is no significant difference in the results when compounds are run singly or in mixtures.

### Method

Membranes (molecular weight cut-off 5000) were first prepared by soaking in the dialysis buffer for a minimum of 1 hour. The dialysis membranes were then mounted into the dialysis cells.

Stock solutions of compounds in dimethylsulphoxide (DMSO) were prepared. This, and all subsequent liquid handling steps, were normally done using a Tecan liquid handling robot. Mixtures of up to five compounds were used. The concentration of each compound in a mixture was normally 1 mM. The mixtures were chosen such that each mixture contains compounds that all have at least a 5 unit difference in molecular weight from one another.

Frozen plasma (EDTA anticoagulant) was normally used for the human plasma binding experiment. The pH of the plasma was adjusted to 7.4 using 1 M HCl immediately before use.

The stock DMSO solution of compounds (7.5 μL) was then added to the dialysis cells along with plasma (750 μl). This was done in duplicate for each mixture. This gave a 1% DMSO in plasma solution with each compound at a concentration of 10 μM (if the stock solution was the standard 1 mM). The dialysis cells were then sealed, secured in a Dianorm rotator unit and equilibrated for 18 hours at 37° C. While the dialysis cells were being equilibrated, the DMSO stock solutions were used for generating optimised HPLC/MS methods for use in the final analysis of the plasma and buffer samples.

After equilibration, the cells were opened and a Tecan liquid handling robot was used to remove aliquots from the plasma and buffer sides of each of the dialysis cells. Blank plasma was then added to the buffer samples and buffer added to the plasma samples such that each sample was in a matrix of 6-fold diluted plasma. Standards were then prepared from the DMSO stock solutions and blank 6-fold diluted plasma. The concentrations of the four standards were normally 50 nM, 150 nM, 500 nM and 2500 nM.

The samples and standards were then analysed using HPLC with MS detection, which allows deconvolution of the mixtures of compounds. The HPLC method involved a forward flushing column switching technique that allows direct injection of the diluted plasma.

### Calculation of Results

The chromatograms were processed using MassLynx software that automatically i 5 calculates a calibration curve for each compound in a mixture and then interpolates the concentrations of buffer and plasma samples. These concentrations still need corrections for the dilution of the plasma. The percentage bound was calculated from the MassLynx data using the following equation:

$$\% bound = 100 - 100 ( \frac{1.2 \times Buffer concentration}{6 \times Plasma concentration} )$$

The factor of 1.2 in the numerator accounts for the small dilution of the aqueous samples with plasma. The factor of 6 in the denominator serves to correct for the 6-fold dilution of the plasma samples with buffer.

The % free (100-% bound) for each compound was calculated from the concentration data, and then recorded.

### Bioavailability (F) in the Rat

This describes the methods used to obtain in vivo pharmacokinetic parameters in the male rat. It is applicable for use with any compound but may need modification based on such parameters as solubility, assay sensitivity, anticipated clearance and half-life, when the default formulation, dose level or sampling intervals may be inappropriate. The method described here represents a standard approach from which justified and documented modifications can be made. This method also allows for single compounds or mixtures (cassettes) to be administered.

### Dose Preparation

A standard dose solution of 1 mg·mL⁻¹ was prepared. The recommended dose vehicle (if the compound was not sufficiently soluble in isotonic saline) was 50% PEG 400:50% sterile water. The required mass of compound was dissolved in the PEG400 before addition of the water. The concentration of the compound in the dose solution was assayed by diluting an aliquot to a nominal concentration of 50 μg·mL⁻¹ and calibrating against duplicate injections of a standard solution and a QC standard at this concentration.

### Dosing

Compounds were administered intravenously as a bolus into a caudal vein to groups of three 250-350 g rats (approximately 1 mL·kg⁻¹). For the oral dose, a separate group of three animals were dosed by oral gavage (3 mL·kg⁻¹). Delivered doses were estimated by weight loss.

Food was not usually withdrawn from animals prior to dosing, although this effect can be investigated if necessary.

### Sample Collection

Pre-dose samples were taken from the oral group. Blood samples (0.25 mL) were taken into 1 ml syringes, transferred to EDTA tubes and plasma was prepared by centrifugation (3 min at 13000 rpm) soon after sample collection.

Sampling Times (min) for the Standard Protocols

| iv | oral |
|---|---|
| 2 | pre |
| 4 | 20 |
| 8 | 40 |
| 15 | 60 |
| 30 | 120 |
| 60 | 180 |
| 120 | 240 |
| 180 | 300 |
| 240 | 360 |
| 300 | — |

### Sample Analysis

The concentration of the analyte(s) were determined in plasma quantitative by mass spectrometry.

### Preparation of Standards and QCs

Standard and quality control stock solutions were prepared at a concentration 50 μg/mL in methanol. The standards and QC stocks were diluted by the TECAN GENESIS and spiked into plasma according to the following table:

| Serial Dilution Program | | | 50 μg/ml stock | |
|---|---|---|---|---|
| | Volume stock | Volume Diluent | Std Conc. | QC Conc. |
| Solution | μL) | (μL) | (ng/mL) | (ng/mL) |
| A | 90 of initial stock | 810 | 1000 | — |
| B | 300 of A | 300 | 500 | 500 |
| C | 300 of B | 300 | 250 | — |
| D | 200 of C | 300 | 100 | 100 |
| E | 300 of D | 300 | 50 | — |
| F | 300 of E | 300 | 25 | — |
| G | 200 of F | 300 | 10 | 10 |
| H | 300 of G | 300 | 5 | — |

10μl of each of the above solutions A-H, produced by serial dilution of the combined standard stock, and 10 μL of solutions B, D and G, produced by serial dilution of the combined QC stock, are added to 96 well 1.2 mL polypropylene tubes containing 50 μL blank plasma by the TECAN. The final concentrations of the standard curve and QC samples produced are shown in the table above. Higher or lower ranges can be obtained using a concentrated or dilute initial stock solution

### Preparation of Samples

To each of the test samples, standards and QCs was added 150 μL of water. The samples were arranged in the order defined below:
1. Standards in order of ascending concentration
2. QCs in order of ascending concentration manual standard.
3. Test samples from IV dosed animals (1M, 2M and then 3M samples)
4. QCs in order of ascending concentration
5. Test samples from PO dosed animals (4M, 5M and then 6M samples)
6. QCs in order of ascending concentration
7. Standards in order of ascending concentration
   The samples were then capped, mixed by repeated inversion and then centrifuged at 3500 rpm in an IEC CENTRA centrifuge for 20 minutes. Aliquots (120 μL) of each sample were analysised LC/MS.

### Mass Spectrometry

A TSQ700 or a TSQ or SSQ7000 mass spectrometer with a HP1100 HPLC system was used. The sources used were APCI or ESI. Standard and quality control samples covering the range of concentrations found in the test samples were expected to be within 25% of the nominal concentration.

### Results

Pharmacokinetic data analysis and tabulation was achieved using WinNonlin and Excel. A standard non-compartmental analysis was used to estimate the parameters tabulated. Bioavailability (F) was calculated from the ratio of the iv and oral AUC (the integral of the plasma concentration time curve) once dose normalised.

### Measurement of Solubility (S)

The solubility of a compound is an important property affecting the preparation of solutions of the compound for screening, as well as influencing absorption of solid doses of the compound in animal and human studies. The method described below for measuring the solubility involves the generation of a saturated solution of the compound, followed by assaying the solution using HPLC with UV quantification and MS identification.

### Method

Saturated solutions for determining the solubility were prepared by placing about 0.3-3.0 ml of solvent in glass screw-top sample tubes along with some of the compound. The tubes are then shaken overnight in the constant temperature room (20° C.). After shaking, undissolved material should be present in the solution, and more was added and shaking continued if this was not the case. The samples were then transferred to a centrifuge tube and centrifuged using a Heraeus Biofuge Fresco centrifuge at 13000 rpm for about 30 minutes. The supernatant was then removed, placed in a new centrifuge tube and centrifuged again for about 30 minutes at 13000 rpm. The undissolved material formed a pellet at the bottom of the tube and the liquid above the pellet was removed for assaying. The solution was then analysed using HPLC with UV quantification. If the response for the compound is very strong then the solution should be accurately diluted such that the response lies within a more suitable range of UV response. A standard was also prepared by accurately weighing a sample of the compound and dissolving it in a suitable volume of a solvent that dissolves it completely (typically, DMSO, ethanol or methanol). This sample was then analysed by HPLC/UV. Again the response of this standard should lie within a suitable range of UV response otherwise a more appropriate concentration should be prepared and analysed by HPLC/UV.

### Results

The solubility (S) was calculated from the observed peak areas in the HPLC/UV chromatograms along with corrections for any dilutions of the sample and differences in injection volumes. The following equation was used:

$$Solubility ( mg \text{/} ml ) = ( \frac{\begin{matrix}Std Conc ( mg \text{/} ml ) \cdot Sample Peak Area \cdot \\ Sample Dilution factor \cdot Std Inj Vol\end{matrix}}{Std Peak Area \cdot Sample Inj Vol} )$$

### REFERENCE EXAMPLE 1

### N-(2-[(2,3-difluorobenzyl)thiol]-6-{[(1R,2S)-2,3-dihydroxy-1-methylpropyl]amino}pyrimidin-4-yl)azetidine-1-sulfonamide

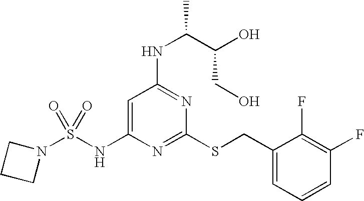 [0286] i) 1-[(4R)-2,2-dimethyl-1,3-dioxolan-4-yl]ethanone

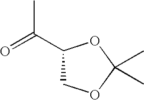

To a solution of (+)-Methyl-(R)-2,2-dimethyl-1,3-dioxolane-4-carboxylate (5 mL) in dry 1:1 diethyl ether/pentane (160 ml) at −115° C. under nitrogen was added 1.6M methyllithium (18 mL) dropwise over 30 min. After further stirring for 1 h 40 min the mixture was quenched with saturated aqueous ammonium chloride solution (80 mL) and then allowed to reach ambient temperature. The organic layer collected and the aqueous layer further exatracted with diethyl ether twice. The organics combined, dried (MgSO₄) and the solvents evaporated in vacuo to give the subtitle compound as a clear oil. Yield: 4.77 g
¹H NMR (300 MHz, CDCl₃): δ 1.40 (s, 3H), 1.47(s, 3H), 2.24(s, 3H), 3.97(m, 1H), 4.19(m, 1H), 4.41(m, 1H)
ii) (1R)-1-[(4S)-2,2-dimethyl-1,3-dioxolan-4-yl]-N-phenylmethyl]ethanamine

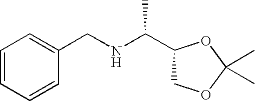

To a solution of the product of step (i) (1-[(4R)-2,2-dimethyl-1,3-dioxolan-4-yl]ethanone) (3.58 g) in dichloroethane (40 mL) was added benzylamine (3 mL) and glacial acetic acid (1.6 mL) followed by cooling the mixture in a ice bath. Sodium triacetoxyborohydride (7.4 g) was added portionwise over 25 min. The mixture then allowed to stir at ambient temperature for 14 h. The mixture was quenched with saturated sodium bicarbonate solution and then extracted with dichloromethane 4 times. The combined organics collected, dried, (MgSO₄) and solvents evaporated to leave a pale yellow oil. Purification by silica gel column chromatography eluting with isohexane/ethyl acetate mixtures from 10 to 20 to 30 to 40% ethylacetate gave the subtitle compound as the first eluting diastereoisomer as a pale yellow oil: Yield 3.66 g
¹NMR (300 MHz, CDCl₃): δ 1.07(d, 3H), 1.36(s, 3H), 1.44(s, 3H), 2.83(quintet, 1H), 3.77(m, 1H), 3.88(, 2H), 4.02(m, 2H), 7.22(m, 1H), 7.35(m, 4H).
iii) (1R)-1-[(4S)-2,2-dimethyl-1,3-dioxolan-4-yl]ethanamine

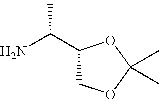

To a solution of product of step (ii) ((1R)-1-[(4S)-2,2-dimethyl-1,3-dioxolan-4-yl]-N-phenylmethyl]ethanamine) (3.65 g) in ethanol (50 mL) was added 10% palladium on charcoal (0.4 g) and the whole hydrogenated at 4 bar at ambient temperature for 12 h. The mixture filtered and the solvent evaporated under vacuo to leave the subtitle compound as a pale yellow oil. Yield: 2.5 g
¹H NMR (300 MHz, CDCl₃): δ 1.07(d, 3H), 1.36(s, 3H), 1.46(s, 3H), 3.08(quintet, 1H), 3.82(m, 1H), 3.93(m, 1H), 3.99(m, 1H)
iv) 6-chloro-2-[(2,3-difluorobenzyl)thio]-N-{(1R)-1-[(4S)-2,2-dimethyl-1,3-dioxolan-4-yl]ethyl}pyrimidin-4-amine

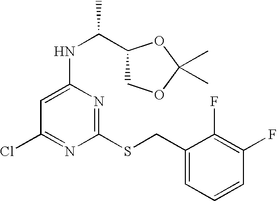

To a solution of product of step (iii) ((1R)-1-[(4S)-2,2-dimethyl-1,3-dioxolan-4-yl]ethanamine) (0.67 g) in acetonitrile (15 mL) was added 4,6-dichloro-2-[(2,3-difluorobenzyl)thio]pyrimidine (WO-2004/011443) (1.3 g), sodium bicarbonate (0.39 g) and the mixture set at reflux under nitrogen for 12 h. The cooled reaction mixture partitioned between ethyl acetate and water. The organic layer collected and the aqueous layer further extracted with ethyl acetate. The combined organics, dried (MgSO₄) and solvent evaporated. The residue purified by silica gel column chromatography eluting with isohexane/ethylacetate mixtures from 5 to 20% ethylacetate to give the subtitle compound as a clear oil. Yield: 1.25 g
¹H NMR (300 MHz, CDCl₃): δ 1.17(d, 3H), 1.34(s, 3H), 1.43(s, 3H), 3.77(dd, 1H), 4.14(m, 2H), 4.37(m, 2H), 5.02(bs, 1H), 6.06(s, 1H), 7.02(m, 2H), 7.26(m, 1H)
v) N-[2-[(2,3-difluorobenzyl)thio]-6-({(1R)-1-[(4S)-2,2-dimethyl-1,3-dioxolan-4-yl]ethyl}amino)pyrimidin-4-yl]azetidine-1-sulfonamide

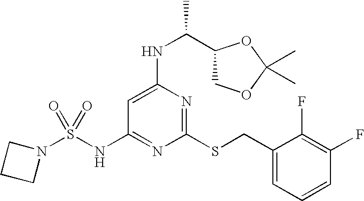

A mixture of product of step (iv) (6-chloro-2-[(2,3-difluorobenzyl)thio]-N-{(1R)-1-[(4S)-2,2-dimethyl-1,3-dioxolan-4-yl]ethyl}pyrimidin-4-amine)) (0.45 g), azetidine-1-sulfonamide (WO-2004/011443) (0.295 g), palladium(II) tris(dibenzylideneacetone) dipalladium (0) (0.1 g), XPhos (0.052 g) and cesium carbonate (0.53 g) in dry dioxane (6 mL) was heated in a microwave in an open vessel at 100° C./300 W max for 15 minutes with stirring. The mixture was allowed to cool to room temperature, acetic acid (2.4 mL) was added and the solvent removed in vacuo. The residues were partitioned between water and ethyl acetate, and the organic fraction was separated, washed with water and brine, dried (MgSO₄), filtered and concentrated in vacuo to give a red gum (1.1 g). The residue purified by silica gel column chromatography eluting with isohexane/ethylacetate mixtures from 5 to 40% ethylacetate to give the subtitle compound as a pale yellow foam. Yield:0.4 g
¹H NMR (300 MHz, DMSO): δ 1.07(d, 3H), 1.26(s, 3H), 1.33(s, 3H), 2.14(quintet, 2H), 3.67(m, 1H), 3.85(t, 4H), 3.94(m, 2H), 4.15(bs, 1H), 4.38(m, 2H), 5.96(s, 1H), 7.14(m, 1H), 7.33(m, 1H), 7.38(m, 1H), 7.46(m, 1H)
vi) N-(2-[(2,3-difluorobenzyl)thio]-6-{[(1R,2S)-2,3-dihydroxy-1-methylpropyl]amino}pyrimidin-4-yl)azetidine-1-sulfonamide

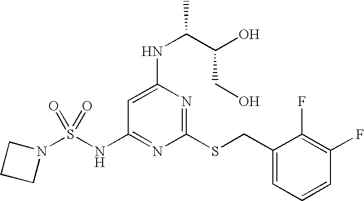

A mixture of the product of step (v) ((N-[2-[(2,3-difluorobenzyl)thio]-6-({(1(1R)-1-[(4S)-2,2-dimethyl-1,3-dioxolan-4-yl]ethyl}amino)pyrimidin-4-yl]azetidine-1-sulfonamide) (0.38 g) and para-toluenesulfonic acid (0.093 g) in methanol (5 mL) and water (3 drops) was heated at 60° C. for 4 h. The solvent was evaporated and the residue taken up in ethyl acetate which was washed with water, dried (MgSO₄) and evaporated to give a pale yellow foam (0.29 g). Purification by trituration with dichloromethane gave the title compound as a off white solid. Yield: 0.23 g
¹H NMR (300 MHz, DMSO): δ 1.04(d, 3H), 2.12(quintet, 2H), 3.30(m, 2H), 3.47(m, 1H), 3.86(m, 4H), 4.17(m, 1H), 4.41(m, 1H), 4.53(bs, 1H), 4.73(bs, 1H), 5.98(bs, 1H), 7.15(m, 1H), 7.32(m, 1H), 7.42(m, 1H), 10.50(bs, 1H)
MS: APCI(+ve) 476 [M+H]⁺

### REFERENCE EXAMPLE 2

### N-(2-[(2,3-difluorobenzyl)thio]-6-{[(1S,2R)-2,3-dihydroxy-1-methylpropyl]amino}pyrimidin-4-yl)azetidine-1-sulfonamide

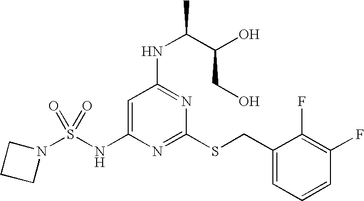 [0306] i) 1-[(4R)-2,2-dimethyl-1,3-dioxolan-4-yl]ethanone

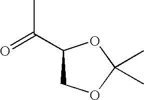

To a solution of (−)-Methyl-(S)-2,2-dimethyl-1,3-dioxolane-4-carboxylate (1 mL) in dry 1:1 diethyl ether/pentane (35 mL) at −115° C. under nitrogen was added 1.6M methyllithium (5.6 mL) dropwise over 10 min. After further stirring for 80 min the mixture was quenched with saturated aqueous ammonium chloride solution (15 mL) and then allowed to reach ambient temperature. The organic layer collected and the aqueous layer further exatracted with diethyl ether twice. The organics combined, dried (MgSO₄) and the solvents evaporated in vacuo to give the subtitle compound as a clear oil. Yield: 0.25 g
¹H NMR (300 MHz, CDCl₃): δ 1.40 (s, 3H), 1.50(s, 3H), 2.25(s, 3H), 4.00(dd, 1H), 4.19(t, 1H), 4.42(dd, 1H)
ii) (1S)-1-[(4R)-2,2-dimethyl-1,3-dioxolan-4-yl]-N-phenylmethyl]ethanamine

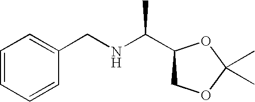

To a solution of the product of step (i) (1-[(4S)-2,2-dimethyl-1,3-dioxolan-4-yl]ethanone) (1.3 g) in dichloroethane (15 mL) was added benzylamine (1.1 mL) and glacial acetic acid (0.575 mL) followed by cooling the mixture in a ice bath. Sodium triacetoxyborohydride (2.68 g) was added portionwise over 25 min. The mixture then allowed to stir at ambient temperature for 14 h. The mixture was quenched with saturated sodium bicarbonate solution and then extracted with dichloromethane 4 times. The combined organics collected, dried, (MgSO₄) and solvents evaporated to leave a pale yellow oil. Purification by silica gel column chromatography eluting with isohexane/ethyl acetate mixtures from 10 to 20 to 30 to 40% ethylacetate gave the subtitle compound as the first eluting diastereoisomer as a clear oil: Yield: 1.1 g
¹H NMR (300 MHz, CDCl₃): δ 1.08(d, 3H), 1.36(s, 3H), 1.42(s, 3H), 1.47(bs, 1H), 2.84(quintet, 1H), 3.77(m, 1H), 3.89(, 2H), 4.03(m, 2H), 7.24(m, 1H), 7.34(m, 4H).
iii) (1S)-1-[(4R)-2,2-dimethyl-1,3-dioxolan-4-yl]ethanamine

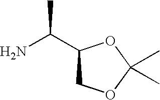

To a solution of product of step (ii) ((1S)-1-[(4R)-2,2-dimethyl-1,3-dioxolan-4-yl]-N-phenylmethyl]ethanamine) (1.4 g) in ethanol (20 mL) was added 10% palladium on charcoal (0.18 g) and the whole hydrogenated at 4 bar at ambient temperature for 12 h. The mixture filtered and the solvent evaporated under vacuo to leave the subtitle compound as a pale yellow oil. Yield: 0.82 g
¹H NMR (300 MHz, CDCl₃): δ 1.06(d, 3H), 1.35(s, 3H), 1.44(s, 3H), 3.06(quintet, 1H), 3.82(m, 1H), 3.96(m, 2H)
iv) 6-chloro-2-[(2,3-difluorobenzyl)thio]-N-{(1S)-1-[(4R)-2,2-dimethyl-1,3-dioxolan-4-yl]ethyl}pyrimidin-4-amine

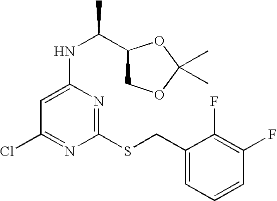

To a solution of product of step (iii) ((1S)-1-[(4R)-2,2-dimethyl-1,3-dioxolan-4-yl]ethanamine) (0.655 g) in acetonitrile (10 mL) was added 4,6-dichloro-2-[(2,3-difluorobenzyl)thio]pyrimidine (WO-2004/011443) (1.2 g), sodium bicarbonate (0.38 g) and is the mixture set at reflux under nitrogen for 12 h. The cooled reaction mixture partitioned between ethyl acetate and water. The organic layer collected and the aqueous layer further extracted with ethyl acetate. The combined organics, dried (MgSO₄) and solvent evaporated. The residue purified by silica gel column chromatography eluting with isohexane/ethylacetate mixtures from 5 to 20% ethylacetate to give the subtitle compound as a clear oil. Yield: 1.5 g
¹H NMR (300 MHz, CDCl₃): δ 1.17(d, 3H), 1.34(s, 3H), 1.43(s, 3H), 3.77(dd, 1H), 4.15(m, 2H), 4.37(m, 2H), 4.98(bs, 1H), 6.06(s, 1H), 7.03(m, 2H), 7.26(m, 1H)
v) N-[2-[(2,3-difluorobenzyl)thio]-6-({(1S)-1-[(4R)-2,2-dimenthyl-1,3-dioxolan-4-yl]ethyl}amino)pyrimidin-4-yl]azetidine-1-sulfonamide

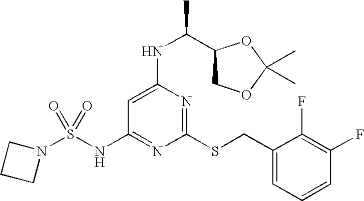

A mixture of product of step (iv) (6-chloro-2-[(2,3-difluorobenzyl)thio]-N-{(1S)-1-[(4R)-2,2-dimethyl-1,3 -dioxolan-4-yl]ethyl}pyrimidin-4-amine)) (0.52 g), azetidine-1-sulfonamide (WO-2004/011443) (0.34 g), palladium(II) tris(dibenzylideneacetone) dipalladium (0) (0.115 g), XPhos (0.06 g) and cesium carbonate (0.612 g) in dry dioxane (8 mL) was heated in a microwave in an open vessel at 100° C./300 W max for 20 minutes with stirring. The mixture was allowed to cool to room temperature, acetic acid (2.4 mL) was added and the solvent removed in vacuo. The residues were partitioned between water and ethyl acetate, and the organic fraction was separated, washed with water and brine, dried (MgSO₄), filtered and concentrated in vacuo to give a red gum (2 g). The residue purified by silica gel column chromatography eluting with isohexane/ethylacetate mixtures from 5 to 40% ethylacetate to give the subtitle compound as a cream foam. Yield:0.42 g
¹H NMR (300 MHz, DMSO): δ 1.04(d, 3H), 1.26(s, 3H), 1.33(s, 3H), 2.14(quintet, 2H), 3.65(m, 1H), 3.85(t, 4H), 3.88(m, 4H), 3.94(m, 2H), 4.38(m, 2H), 5.96(s, 1H), 7.13(m, 1H), 7.33(m, 1H), 7.38(m, 1H), 7.46(m, 1H), 10.56 (bs, 1H)
vi) N-(2-[(2,3-difluorobenzyl)thio]-6-{[(1S,2R)-2,3-dihydroxy-1-methylpropyl]amino}pyrimidin-4-yl)azetidine-1-sulfonamide

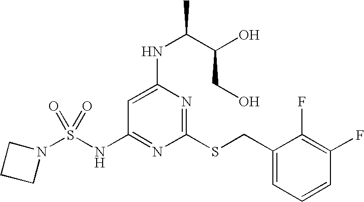

A mixture of the product of step (v) ((N-[2-[(2,3-difluorobenzyl)thio]-6-({(1S)-1-[(4R)-2,2-dimethyl-1,3-dioxolan-4-yl]ethyl}amino)pyrimidin-4-yl]azetidine-1-sulfonamide) (0.31 g) and para-toluenesulfonic acid (0.076 g) in methanol (5 mL) and water (3 drops) was heated at 60° C. for 4.5 h. The solvent was evaporated and the residue taken up in ethyl acetate which was washed with water, dried (MgSO₄) and evaporated to give a pale yellow foam. Purification by silica gel chromatography eluting with dichloromethane/methanol mixtures (1 to 2% methanol) followed by trituration with dichloromethane gave the title compound as a white solid. Yield: 0.185 g
¹H NMR (300 MHz, DMSO): δ 1.07(d, 3H), 2.13(quintet, 2H), 3.23(m, 2H), 3.46(m, 1H), 3.87(t, 4H), 4.23(bs, 1H), 4.39(q, 1H), 4.50(bs, 1H), 4.76(bs, 1H), 6.02(bs, 1H), 7.15(m, 1H), 7.22(bs, 1H), 7.33(m, 1H), 7.44(t, 1H), 10.49(bs, 1H)
MS: APCI(+ve) 476 [M+H]⁺

### REFERENCE EXAMPLE 3

### N-(2-[(2,3-difluorobenzyl)thio]-6-{[(1S,2S)-2,3-dihydroxy-1-methylpropyl]amino}pyrimidin-4-yl)azetidine-1-sulfonamide

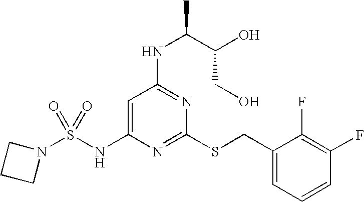 [0326] i) 1-[(4R)-2,2-dimethyl-1,3-dioxolan-4-yl]ethanone

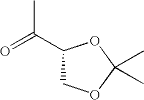

To a solution of (+)-Methyl-(R)-2,2-dimethyl-1,3-dioxolane-4-carboxylate (5 mL) in dry 1:1 diethyl ether/pentane (160 ml) at −115° C. under nitrogen was added 1.6M methyllithium (18 mL) dropwise over 30 min. After further stirring for 1 h 40 min the mixture was quenched with saturated aqueous ammonium chloride solution (80 mL) and then allowed to reach ambient temperature. The organic layer collected and the aqueous layer further exatracted with diethyl ether twice. The organics combined, dried (MgSO₄) and the solvents evaporated in vacuo to give the subtitle compound as a clear oil. Yield: 4.77 g
¹H NMR (300 MHz, CDCl₃): δ 1.40 (s, 3H), 1.47(s, 3H), 2.24(s, 3H), 3.97(m, 1H), 4.19(m, 1H), 4.41(m, 1H)
ii) (1S)-1-[(4S)-2,2-dimethyl-1,3-dioxolan-4-yl]-N-phenylmethyl]ethanamine

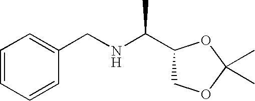

To a solution of the product of step (i) (1-[(4R)-2,2-dimethyl-1,3-dioxolan-4-yl]ethanone) (3.58 g) in dichloroethane (40 mL) was added benzylamine (3 mL) and glacial acetic acid (1.6 mL) followed by cooling the mixture in a ice bath. Sodium triacetoxyborohydride (7.4 g) was added portionwise over 25 min. The mixture then allowed to stir at ambient temperature for 14 h. The mixture was quenched with saturated sodium bicarbonate solution and then extracted with dichloromethane 4 times. The combined organics collected, dried, (MgSO₄) and solvents evaporated to leave a pale yellow oil. Purification by silica gel column chromatography eluting with isohexane/ethyl acetate mixtures from 10 to 20 to 30 to 40% ethylacetate gave the subtitle compound as the second eluting diastereoisomer as a pale yellow oil: Yield 0.74 g
¹H NMR (300 MHz, CDCl₃): δ 1.02(d, 3H), 1.36(s, 3H), 3.38(s, 3H), 2.80(bs, 1H), 2.76(quintet, 2H), 3.68(m, 2H), 3.96(m, 1H), 7.22(m, 1H), 7.35(m, 4H),
iii) (1S)-1-[(4S)-2,2-dimethyl-1,3-dioxolan-4-yl]ethanamine

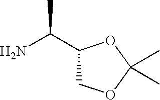

To a solution of product of step (ii) ((1S)-1-[(4S)-2,2-dimethyl-1,3-dioxolan-4-yl]-N-phenylmethyl]ethanamine) (0.73 g) in ethanol (20 mL) was added 10% palladium on charcoal (0.1 g) and the whole hydrogenated at 4 bar at ambient temperature for 12 h. The mixture filtered and the solvent evaporated in vacuo to leave the subtitle compound as a pale yellow oil. Yield: 0.43 g
¹H NMR (300 MHz, CDCl₃): δ 1.00(d, 3H), 1.35(s, 3H), 1.43(s, 3H), 2.87(quintet, 1H), 3.63(t, 1H), 3.78(m, 1H), 4.03(m, 1H)
iv) 6-chloro-2-[(2,3-difluorobenzyl)thio]-N-{(1S)-1-[(4S)-2,2-dimethyl-1,3-dioxolan-4-yl]ethyl}pyrimidin-4-amine

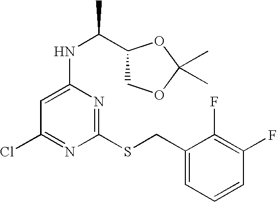

To a solution of product of step (iii) ((1S)-1-[(4S)-2,2-dimethyl-1,3-dioxolan-4-yl]ethanamine) (0.32 g) in acetonitrile (8 mL) was added 4,6-dichloro-2-[(2,3-difluorobenzyl)thio]pyrimidine (WO-2004/011443) (0.616 g), sodium bicarbonate (0.185 g) and the mixture set at reflux under nitrogen for 12 h. The cooled reaction mixture partitioned between ethyl acetate and water. The organic layer collected and the aqueous layer further extracted with ethyl acetate. The combined organics, dried (MgSO₄) and solvent evaporated. The residue purified by silica gel column chromatography eluting with isohexane/ethyl acetate mixtures from 5 to 20% ethyl acetate to give the subtitle compound as a clear oil. Yield:0.58 g
¹H NMR (300 MHz, CDCl₃): δ 1.23(d, 3H), 1.36(s, 3H), 1.44(s, 3H), 3.58(t, 1H), 3.98(t, 2H), 4.14(m, 1H), 4.37(s, 2H) 5.07(bs, 1H), 6.05(s, 1H), 7.02(m, 2H), 7.30(m, 1H)
v) N-[2-[(2,3-difluorobenzyl)thio]-6-({(1S)-1-[(4S)-2,2-dimethyl-1,3-dioxolan-4-yl]ethyl}amino)pyrimidin-4-yl]azetidine-1-sulfonamide

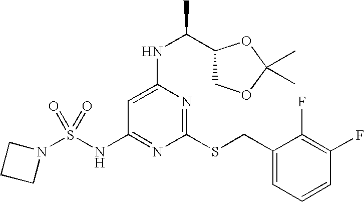

A mixture of product of step (iv) (6-chloro-2-[(2,3-difluorobenzyl)thio]-N-{(1S-1-[(4S)-2,2-dimethyl-1,3-dioxolan-4-yl]ethyl}pyrimnidin-4-amine)) (0.37 g), azetidine-1-sulfonamide (WO-2004/011443) (0.24 g), palladium(II) tris(dibenzylideneacetone) dipalladium (0) (0.082 g), XPhos (0.042 g) and cesium carbonate (0.435 g) in dry dioxane (5 mL) was heated in a microwave in an open vessel at 100° C./300 W max for 15 minutes with stirring. The mixture was allowed to cool to room temperature, acetic acid (2.4 mL) was added and the solvent removed in vacuo. The residues were partitioned between water and ethyl acetate, and the organic fraction was separated, washed with water and brine, dried (MgSO₄), filtered and concentrated in vacuo to give a red gum (1.1 g). The residue purified by silica gel column chromatography eluting with isohexane/ethylacetate mixtures from 10 to 40% ethyl acetate to give the subtitle compound as a pale yellow foam. Yield:0.36 g
¹H NMR (300 MHz, CDCl₃): δ 1.24(d, 3H), 1.36(s, 3H), 1.45(s, 3H), 2.26(quintet, 2H), 3.62(t, 1H), 3.95(t, 1H), 3.99(m, 4H), 4.27(m, 1H), 4.34(m, 2H), 5.06(bs, 1H), 5.92(s, 1H), 7.02(m, 2H), 7.23(m, 1H), 7.38(m, 1H), 7.46(m, 1H)
vi) N-(2-[(2,3-difluorobenzyl)thio]-6-{[(1S,2S)-2,3-dihydroxy-1-methylpropyl]amino}pyrimidin-4-yl)azetidine-1-sulfonamide

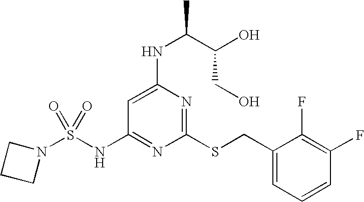

A mixture of the product of step (v) ((N-[2-[(2,3-difluorobenzyl)thio]-6-({(1S)-1-[(4S)-2,2-dimethyl-1,3-dioxolan-4-yl]ethyl}amino)pyrimidin-4-yl]azetidine-1-sulfonamide) (0.346 g) and para-toluenesulfonic acid (0.084 g) in methanol (5 mL) and water (2 drops) was heated at 60° C. for 3 h. The solvent was evaporated and the residue taken up in ethyl acetate which was washed with water, dried (MgSO₄) and evaporated to give a pale yellow foam.

Purification by silica gel chromatography eluting with dichloromethane/methanol mixtures (2 to 4% methanol) followed by trituration with dichloromethane gave the title compound as a white solid. Yield: 0.185 g
¹H NMR (300 MHz, CDCl₃): δ 1.27(d, 3H), 2.26(quintet, 2H), 3.56(m, 2H), 3.71(m, 1H), 3.96(m, 4H), 4.17(t, 4H), 4.25(m, 1H), 4.35(s, 2H), 5.14(bd, 1H), 6.01(s, 1H), 7.06(m, 2H), 7.23(m, 1H)
MS: APCI(+ve) 476 [M+H]⁺

1. A compound of formula (1) 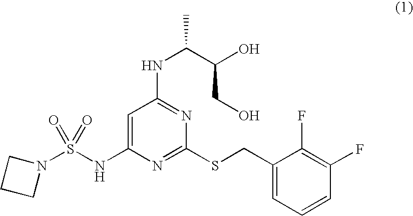
or a pharmaceutically acceptable salt thereof.

2. A compound according to claim 1 or a pharmaceutically acceptable salt thereof for use in the treatment of a chemokine mediated disease or condition.

3. A compound according to claim 2 or a pharmaceutically acceptable salt thereof for use as a medicament for the treatment of asthma, allergic rhinitis, COPD, inflammatory bowel disease, osteoarthritis, osteoporosis, rheumatoid arthritis, or psoriasis.

4. A pharmaceutical composition comprising a compound according to claim 1 or a pharmaceutically acceptable salt thereof together with a pharmaceutically-acceptable diluent or carrier.

5. A process for the preparation of a compound according to claim 1 or a pharmaceutically acceptable salt thereof, which comprises:
(a) treating a compound of formula (2a) 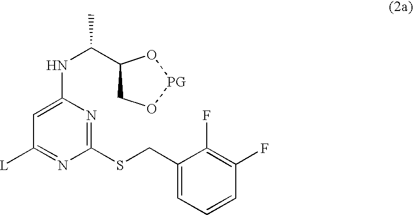
wherein PG is either a protecting group or two separate hydrogen atoms and L is a leaving group, with a sulfonamide of formula (2c) 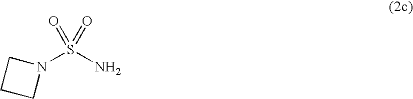
in the presence of a suitable base, catalyst and solvent, and optionally thereafter (i) and/or (ii) in any order:
i) removing any protecting groups;
ii) forming a salt;
or alternatively
(b) treating a compound of formula (2b) 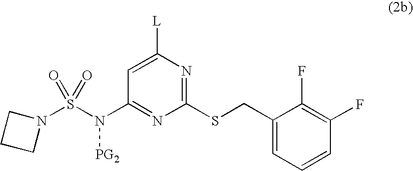
wherein PG2 is a protecting group and L is a leaving group with an amine of formula (2d) 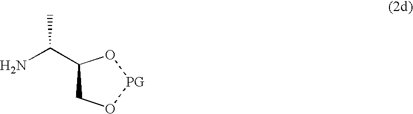
wherein PG is a protecting group or two separate hydrogen atoms,
in the presence of a suitable base, and solvent, and optionally thereafter (i) and/or (ii) in any order:
i) removing any protecting groups,
ii) forming a salt.

6. A compound of the formula (1a) 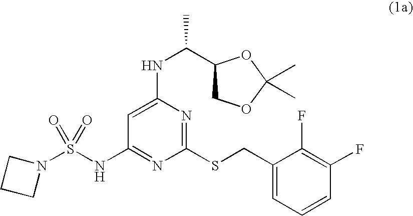
and pharmaceutically acceptable salts thereof.

7. A compound of formula (2a) wherein L is halogen.

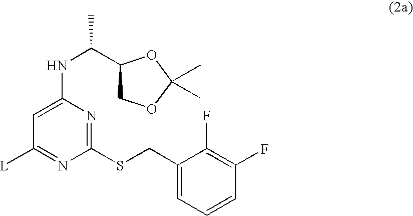

8. A compound of the formula (2e) wherein L is halogen

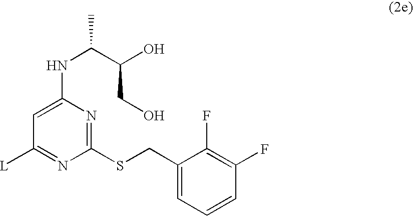

9. A combination therapy which comprises administering a compound of formula (1) as defined in claim 1 or a pharmaceutically acceptable salt thereof, or a pharmaceutical composition or formulation comprising a compound of formula (1), concurrently or sequentially with other therapy and/or another pharmaceutical agent.

10. A combination therapy as claimed in claim 9 for the treatment of asthma, allergic rhinitis, COPD, inflammatory bowel disease, irritable bowel syndrome, osteoarthritis, osteoporosis, rheumatoid arthritis, or psoriasis.

11. A pharmaceutical composition which comprises a compound of formula (1) or a pharmaceutically acceptable salt thereof, in conjunction with another pharmaceutical agent.

12. A pharmaceutical composition as claimed in claim 16 for the treatment of asthma, allergic rhinitis, COPD, inflammatory bowel disease, irritable bowel syndrome, osteoarthritis, osteoporosis, rheumatoid arthritis, or psoriasis.

13. A pharmaceutical composition as claimed in claim 11 for the treatment of cancer.

14. A compound as claimed in claim 1 or a pharmaceutically acceptable salt thereof in any one of the following crystalline forms:
(a) as characterised by an X-ray powder diffraction (XRPD) pattern as shown in Table 3 herein, assigned as modification A;
(b) as characterised by an X-ray powder diffraction (XRPD) pattern as shown in Table 4 hereinbefore, assigned as modification B;
(c) as characterised by an X-ray powder diffraction (XRPD) pattern as shown in Table 5 herein, assigned as modification C;
(d) as characterised by an X-ray powder diffraction (XRPD) pattern as shown in Table 6 herein, assigned as modification D;
(e) as characterised by an X-ray powder diffraction (XRPD) pattern as shown in Table 7 herein, assigned as modification E; or
(f) as characterised by an X-ray powder diffraction (XRPD) pattern as shown in Table 8 herein, assigned as modification F.
